(19) United States
(12) Patent Application Publication
(10) Pub. No.: US 2026/0066040 A1
(43) Pub. Date: Mar. 5, 2026

(54) SYSTEMS AND METHODS FOR PHASING MUTATIONS IN TUMORS

(71) Applicant: Genentech, Inc., South San Francisco, CA (US)

(72) Inventors: Patrick Michael LEBLANC, Durham, NC (US); Daniel Gregory OREPER, San Francisco, CA (US); Andrew John WALLACE, Hayward, CA (US); Suchit Sushil JHUNJHUNWALA, Foster City, CA (US)

(21) Appl. No.: 19/385,942

(22) Filed: Nov. 11, 2025

Related U.S. Application Data
(63) Continuation of application No. PCT/US2024/029243, filed on May 14, 2024.
(60) Provisional application No. 63/502,387, filed on May 15, 2023.

(51) Int. Cl. G16B 20/10; G16B 5/20; G16B 15/30; G16B 30/00; G16B 45/00

(52) U.S. Cl. CPC G16B 20/10; G16B 5/20; G16B 15/30; G16B 30/00; G16B 45/00

(57) ABSTRACT

This application relates generally to analyzing mutations in tumors, and more particularly, to systems and methods for phasing mutations in tumors of subjects (e.g., cancer patients). An exemplary method for phasing mutations in a tumor of a subject comprises enumerating, based on tumor DNA and/or RNA sequence reads, a set of unique mutation patterns observed in the plurality of sequence reads; counting the set of unique patterns observed in the sequence reads to calculate a quantity of each of the unique mutation patterns and/or a quantity of each combination of unique mutation pattern and a transcript group; determining mutation pattern probabilities; and inputting the mutation pattern quantities and the mutation pattern probabilities into a statistical model to estimate at least one of a set of haplotype-existence probabilities that each of the haplotypes exists, a set of haplotype prevalences, and a set of haplotype-transcript prevalences.

### CROSS-REFERENCE TO RELATED APPLICATIONS

This application is a continuation of International Application No. PCT/US2024/029243, filed on May 14, 2024, which claims the priority benefit of U.S. Provisional Patent Application Ser. No. 63/502,387, filed on May 15, 2023, the contents of which are incorporated herein by reference in their entirety.

### TECHNICAL FIELD

This application relates generally to analyzing mutations in tumors, and more particularly, to systems and methods for phasing mutations in tumors of subjects (e.g., cancer patients).

### BACKGROUND

Cancer immunology and the advent of next-generation sequencing (NGS) and bioinformatics techniques have advanced the development of individualized neoantigen-specific immunotherapies. Individualized neoantigen-specific immunotherapies may include any treatments or immunotherapies that are designed and developed based on each patient's particular tumor mutations with the aim of inducing, for example, high-affinity T-cell responses against cancer cells. Specifically, while tumor cells share a majority of their DNA with healthy cells, tumor cells also carry unique mutations. Genetic mutations can lead to the expression of unique tumor antigens called neoantigens. Neoantigens are mutant proteins whose fragments are presented via major histocompatibility complex (MHC) to T cells and thereby potentially drive anti-tumor immunity. Accordingly, neoantigens have emerged as a promising target for immunotherapies that try to induce the immune system to specifically destroy tumor cells.

Short-read DNA (and/or RNA) sequencing can be used to identify candidate neoantigens. Specifically, DNA can be extracted from an individual patient's tumor tissues and sequenced. Further, DNA can also be extracted from one or more matched-normal tissue samples from that same patient and sequenced, or alternately from a normal tissue sample collected from one or more comparable individuals. Both tumor and normal short-read sequencing data can be aligned to the reference genome. If multiple aligned reads from the tumor sample do not match the reference genome at some position, but most aligned reads from the normal sample(s) do match the reference genome at that same position, this may be indicative of a somatic tumor mutation at that position. Such somatic mutations can be detected through somatic mutation callers which may use some type of statistical approach. As part of somatic mutation calling, the “variant allele frequency” (VAF) of a mutant allele can be estimated. A high VAF typically corresponds to a mutation which is more clonal, and accordingly the corresponding peptide or protein (e.g., neoantigen) may be a better immunotherapy target as more cells in the tumor can be targeted. If multiple aligned reads from the normal sample do not match the reference genome at some position, this may be indicative of a germline variant that is present in at least some cells in normal tissue, and likely present in some cells in tumor tissue as well. These variants can be detected through germline variant callers. RNA reads can similarly be used to identify somatic and germline variants. Regardless of the called-variant source, somatic and germline variants can be then projected into the reference transcriptome (as specified in, for example, a gene transfer format (GTF) file) and translated in silico (using, e.g., Ensembl Variant Effect Predictor (VEP) and known codon triplet-to-amino acid correspondences) to predict tumor peptides. The mutant subsequences of tumor polypeptides (arising from somatic mutations) correspond to predicted candidate neoantigens.

Somatic and germline variant calling only determines the position of somatic and germline variants that have, in aggregate, occurred in at least one copy of a gene in some cell that was sequenced. However, whether using bulk or single-cell sequencing (and more broadly than the context of tumor sequencing), multiple copies of a given gene are usually present in a given sample/cell to be sequenced, and mutation calling does not by itself reveal which subsets of somatic and germline variant alleles have co-occurred in any single one of these gene copies. In other words, some copies of a gene may contain one subset of called variant alleles, whereas other copies of that gene may contain a different subset of variant alleles—and the sequencing data from every copy of the gene is typically mixed together. The combinations of variant alleles that co-occur are often described as haplotypes. Determining the variant alleles that co-occur in at least one copy of a gene (or one copy of a subregion of the gene) is referred to as “phasing” variants. In the context of phasing somatic variants, phasing variants (also referred to as “mutation phasing”) is required in order to accurately perform in-silico translation, and in turn correctly predict candidate neoantigens. Further, these mutations may need to be phased together with germline variants because most of the variants near a given somatic mutation can be germline variants.

While variant phasing (somatic or germline) can be accomplished directly through certain complementary experiments, such approaches are low-throughput techniques that are not practical in clinical settings. On the other hand, a variety of computational approaches can phase germline variants (not somatic variants) and identify haplotypes present in a given sample from short-read NGS (or microarray) data. Some approaches for germline variant phasing employ population-reference-based phasing: these approaches exploit the fact that shared ancestry and limited recombination give rise to shared and inherited haplotype blocks; any new patient's haplotypes are assumed to be a mosaic of haplotype blocks from a reference panel of known haplotypes. Other phasing techniques may avoid reference population assumptions and restrict themselves to ‘read-backed’ computational phasing. Specifically, read-backed phasing may serve to identify haplotypes based on high-throughput sequence reads that overlap more than one mutation.

Unfortunately, the overwhelming majority of existing computational techniques—whether reference-based and/or read-backed—are only appropriate for phasing germline variants in healthy tissue, because they rely on assumptions that do not hold in tumor sequencing data. Reference-based techniques that expect inheritance of haplotype blocks are clearly unsuitable because somatic mutations—by definition—are not inherited. But more generally, most methods for computational phasing expect no more than two unique haplotypes to exist for a given gene—which is not the case when dealing with bulk data in which cells from multiple tumor cell subpopulations within the same patient may be sequenced. Nor is it necessarily the case in single-cell sequencing of tumor cells, in which some cells may have undergone copy number amplifications and subsequent mutations of the given gene. A smaller fraction of existing methods can phase polyploid genomes (e.g., genomes comprising more than two haplotypes), but these methods often still assume that each haplotype is equally represented in the genomes of the sequenced samples—an assumption which is again subverted by the presence of tumor subpopulations and copy number variation. For similar reasons, these polyploid phasing methods are inappropriate for RNA sequence read data, as haplotypes-even if present in equal proportion in the genomic DNA—can be expressed at different rates.

Most algorithms phase germline mutations in diploid samples. For example, some algorithms involve the combination of graph and read-backed phasing, such as Hapcut2 and “max-cut” bipartition of mutations. As another example, some algorithms (e.g., Shapeit4) involve the combination of population reference and hidden Markov model (HMM) phasing, where each individual is assumed to be a mosaic of reference haplotypes. There are far fewer methods for polyploid phasing, and these are generally suboptimal for phasing tumor sequencing data. For example, Haptree would be ill-suited for use with bulk tumor sequencing data as it assumes equal dosage of haplotypes (i.e., that the haplotypes are present in equal proportions) in assigning sequence reads to each haplotype. Moreover, this assumption is inappropriate for modeling RNA sequencing data or for accounting for copy number variations (CNVs). Alternately, Hapcompass is purely graph-based and does not make such assumptions, but at the same time typically underperforms in normal polyploid phasing tasks relative to newer methods.

In addition to the polyploid nature and unequal haplotype/expression dosage inherent to tumor samples, phasing is especially hindered—for normal samples as well—by short-read limitations. Most short reads will not unambiguously indicate which haplotypes are present in the observed samples, as they are not long enough to cover all variants in a gene simultaneously. Further, as part of clinical sequencing, T-N-R triplets of sequencing data (i.e., Tumor DNA—Normal DNA—RNA sequencing data), rather than a single sequencing sample that other phasing methods are intended for, may be obtained. Thus, new systems and methods suitable for phasing mutations in tumors of cancer patients are desirable.

### SUMMARY

Described herein are methods, systems, and programming for phasing mutations (a catch-all term for phasing somatic and/or germline variants) identified in a tumor of a subject. The overarching idea is that certain types of observed DNA and/or RNA sequence reads are more likely to be generated by some haplotypes (and for RNA sequence reads, by some transcripts) and not others—where a haplotype is defined as a given combination of variant alleles (e.g., single nucleotide variants (SNVs), indels, etc.) that co-occur in at least one copy of a gene or gene subregion (where, again, the term “variants” in this context may refer to germline variants and/or somatic mutations). As such, DNA and/or RNA sequence reads provide evidence for the existence of certain haplotypes, and/or the prevalence of certain haplotypes, and/or (if RNA sequence read data is available) for the level of expression of certain haplotypes in certain transcripts.

In some embodiments, a plurality of sequence reads derived from tumor cells obtained from the subject may be accessed. The plurality of sequence reads (e.g., single reads or paired-end reads) may comprise tumor DNA sequence reads and/or tumor RNA sequence reads. A set of unique mutation patterns (i.e., all possible unique combinations of observed mutation alleles that may occur in a given gene sequence or portion thereof) may be enumerated based on a set of mutations (e.g., tumor-associated mutations) identified in the plurality of sequence reads (where a given mutation allele may or may not be observed in a given individual sequence read). A quantity for each of the unique mutation patterns may then be calculated by counting the number of times that each unique mutation pattern of the set is observed in the plurality of sequence reads. For each of the unique mutation patterns, and for each haplotype in the set of all candidate haplotypes (where the candidate haplotype set is the set of all possible combinations of observable variant alleles, irrespective of the actual sequence read data (e.g., for biallelic mutations, a set containing 2\hat{ }(num_mutations) haplotypes)), the probability that a hypothetical DNA sequence read from that haplotype will exhibit the unique mutation pattern may be determined. Similarly, if RNA sequence reads are available, for each possible unique mutation pattern, for each possible unique transcript group (described shortly), for each candidate haplotype, and for each transcript, a probability that a hypothetical RNA sequence read from the given haplotype and transcript will exhibit the unique mutation pattern and simultaneously be consistent with the given unique transcript group can be determined (i.e., where each unique transcript group is one of the non-empty subsets of the transcripts that are transcribed by a gene to be phased; i.e., there are 2\hat{ }(number of gene transcripts−1) transcript groups; a base within a sequence read is consistent with a transcript group if that base aligns to a position within an exon that is solely expressed by the transcripts in that transcript group and no other transcript; a sequence read is consistent with the smallest transcript group with which its bases are consistent).

Each combination of haplotype and transcript may generate RNA sequence reads that potentially exhibit a given combination of mutation pattern and a transcript group associated with a given gene. Combinations of haplotype and transcript thus correspond to the underlying genomic reality. Mutation patterns and transcript groups correspond to noisy and/or ambiguous observations (in the form of DNA or RNA sequence reads) of the underlying reality. Each combination of haplotype and transcript can yield multiple different combinations of mutation pattern and transcript group. Each combination of mutation pattern and transcript group could have arisen from multiple pairwise combinations of haplotype and transcript. As such, the correspondence between pairwise combinations of haplotype and transcript) and pairwise combinations of mutation pattern and transcript group is a many-to-many relationship.

The unique mutation pattern quantities and the mutation pattern probabilities determined for DNA sequence reads, and/or the unique mutation pattern-transcript group quantities and mutation pattern-transcript group probabilities determined for RNA sequence reads may then be input into a statistical model to estimate at least one of: (i) a set of haplotype-existence probabilities (i.e., probabilities that each of the haplotypes is present in the sample), (ii) a set of haplotype prevalences (i.e., the prevalence of each haplotype in the set of identified haplotypes, expressed as, e.g., the percentage of the sum total number of DNA molecules in the sequenced sample that are of each haplotype), and/or (iii) a set of haplotype-transcript prevalences (i.e., the prevalence of each haplotype-transcript combination in the set of identified haplotype-transcript combinations expressed as, e.g., the percentage of the sum total number of RNA molecules in the sequenced sample that are of each haplotype-transcript combination). The disclosed methods can be performed using DNA sequence reads, RNA sequence reads, or a combination of DNA sequence reads and RNA sequence reads if they are both available.

In some embodiments, the set of haplotype-existence probabilities, the set of haplotype prevalences, and/or the set of haplotype-transcript prevalences determined by the statistical model are used to identify a set of tumor-associated peptides or proteins (e.g., neoantigens) and/or to select a subset of those tumor-associated peptides or proteins for use in developing patient-specific therapies (e.g., anti-cancer therapies). For example, a set of haplotypes identified in the sequence read data (e.g., based on comparison of haplotype-existence probabilities to a predefined threshold) can be translated in silico to determine tumor-associated peptide or protein sequences (e.g., by parsing nucleotide sequence data into codons, and then translating the codons into a corresponding amino acid sequence). Haplotype prevalence data output by the statistical model can be used to rank-order and/or select a subset of the tumor-associated peptide or protein sequences for use in developing patient-specific therapies (e.g., anti-cancer therapies). Haplotype-transcript prevalence data output by the statistical model can be used to further refine the rank-ordering and/or selection of the subset of the tumor-associated peptide or protein sequences for use in developing patient-specific therapies (e.g., anti-cancer therapies), for example, by determining that the transcript expression for one tumor-associated peptide or protein (and its associated haplotype) is much more abundant than that same transcript's expression for a different tumor-associated peptide or protein (and its different associated haplotype)

Some embodiments of the present disclosure include a system including one or more data processors. In some embodiments, the system includes a non-transitory computer readable storage medium containing instructions which, when executed on the one or more data processors, cause the one or more data processors to perform the steps of any method disclosed herein.

Some embodiments of the present disclosure include a computer-program product tangibly embodied in a non-transitory machine-readable storage medium, including instructions configured to cause one or more data processors to perform the steps of any method disclosed herein.

Some embodiments of the present disclosure include a vaccine including one or more peptides, a plurality of nucleic acids that encode the one or more peptides, or a plurality of cells expressing the one or more peptides, wherein the one or more peptides are selected from among a set of peptides identified by performing the steps of any method disclosed herein.

Some embodiments of the present disclosure include a method of designing a vaccine including one or more peptides, a plurality of nucleic acids that encode the one or more peptides, or a plurality of cells expressing the one or more peptides, the method comprising: identifying the one or more peptides using a method as described herein.

Some embodiments of the present disclosure include a method of manufacturing a vaccine including producing a vaccine including one or more peptides, a plurality of nucleic acids that encode the one or more peptides; or a plurality of cells expressing the one or more peptides, wherein the one or more peptides are selected from among a set of peptides identified by performing the steps of any method disclosed herein.

Some embodiments of the present disclosure include a pharmaceutical composition comprising one or more peptides selected from among a set of peptides identified by performing the steps of any method disclosed herein.

Disclosed herein are methods for phasing mutations identified in a tumor of a subject, comprising, by one or more computing devices: accessing a plurality of sequence reads derived from tumor cells obtained from the subject, wherein the sequence reads comprise tumor DNA sequence reads and/or tumor RNA sequence reads; enumerating a set of unique mutation patterns observed in the plurality of sequence reads; counting a number of sequence reads that exhibit each unique mutation pattern of the set of unique mutation patterns observed in the sequence reads to calculate a quantity of each of the unique mutation patterns, and/or counting a number of sequence reads that exhibit each combination of a unique mutation pattern of the set of unique mutation patterns and a transcript group from one or more transcript groups associated with a gene to calculate a quantity of each combination of unique mutation pattern and transcript group; determining, for each of the unique mutation patterns, a probability, for each haplotype of a set of haplotypes, that a hypothetical DNA sequence read from the haplotype will exhibit the unique mutation pattern, and/or a probability, for each combination of a haplotype from the set of haplotypes, a transcript from a set of transcripts associated with the gene, and a transcript group from a set of transcript groups associated with the gene, that a hypothetical RNA sequence read from the haplotype and the transcript will exhibit the unique mutation pattern and the transcript group; inputting the unique mutation pattern quantities and/or unique mutation pattern-transcript group quantities, and the unique mutation pattern probabilities and/or unique mutation pattern-transcript group probabilities into a statistical model to estimate at least one of (i) a set of haplotype-existence probabilities that each of the haplotypes exists, (ii) a set of haplotype prevalences, and (iii) a set of haplotype-transcript prevalences; and outputting at least one of (i) the set of haplotype-existence probabilities, (ii) the set of haplotype prevalences, and (iii) the set of haplotype-transcript prevalences.

In some embodiments, the estimation of at least one of the set of haplotype-existence probabilities, the set of haplotype prevalences, and the set of haplotype-transcript prevalences comprises: (i) using the statistical model to sample from at least one of a haplotype-existence posterior probability distribution, a haplotype prevalence posterior probability distribution, and a haplotype-transcript prevalence posterior probability distribution for each haplotype of the set; and (ii) calculating point estimates for haplotype-existence probability, haplotype prevalence, and haplotype-transcript prevalence for each haplotype of the set using the samples from the respective posterior probability distributions.

In some embodiments, the method further comprises: identifying a set of mutant peptide sequences using in silico translations of one or more haplotype sequences and/or haplotype transcripts based on the set of haplotype-existence probabilities, the set of haplotype prevalences, and/or the set of haplotype-transcript prevalences. In some embodiments, the one or more haplotype sequences and/or haplotype transcripts are associated with non-zero haplotype-existence probabilities.

In some embodiments, the method further comprises: selecting one or more mutant peptide sequences from the set of mutant peptide sequences using one or more predetermined criteria comprising a predetermined criterion that applies to the set of haplotype prevalences and/or the set of haplotype-transcript prevalences. In some embodiments, the one or more predetermined criteria include a predefined haplotype prevalence threshold and/or a predetermined haplotype-transcript threshold.

In some embodiments, the method further comprises selecting one or more mutant peptide sequences from the set of mutant peptide sequences by ranking of the set of peptide sequences based on the set of haplotype prevalences and/or the set of haplotype-transcript prevalences.

In some embodiments, the method further comprises: generating, by a machine-learning model, a prediction of a likelihood of presentation in a major histocompatibility complex (MHC) for one or more of the set of mutant peptide sequences and/or a prediction of an immunogenicity for one or more of the set of mutant peptide sequences.

In some embodiments, accessing the sequence reads further comprises accessing a plurality of normal DNA sequence reads derived from healthy cells obtained from the subject. In some embodiments, counting the set of unique mutation patterns observed in the plurality of sequence reads further comprises: calculating a quantity of each unique mutation pattern in the normal DNA sequence reads.

In some embodiments, counting the set of unique mutation patterns observed in the plurality of sequence reads further comprises: calculating a quantity of each unique mutation pattern in the tumor DNA sequence reads; and/or calculating a quantity of each unique mutation pattern and transcript-group combination in the tumor RNA sequence reads.

In some embodiments, the probability that the hypothetical RNA sequence read from that haplotype and transcript will exhibit the unique mutation pattern and the transcript group is calculated from a haplotype-transcript prevalence, a conditional probability of observing the combination of unique mutation pattern and the transcript group in an RNA sequence read, and a transcript length. In some embodiments, calculation of the probability that the hypothetical RNA sequence read from that haplotype and transcript will exhibit the unique mutation pattern and the transcript group further comprises accounting for a probability of miscalling tumor RNA sequence reads with the unique mutation pattern.

In some embodiments, the probability that the hypothetical DNA sequence read from that haplotype will exhibit the unique mutation pattern is calculated from a haplotype prevalence and a conditional probability of observing the unique mutation pattern in a DNA sequence read. In some embodiments, the calculation of the probability that the hypothetical DNA sequence read from that haplotype will exhibit the unique mutation pattern further comprises accounting for a probability of miscalling tumor DNA sequence reads with the unique mutation pattern.

In some embodiments, calculation of the probability that the hypothetical RNA sequence from that haplotype and transcript will exhibit the unique mutation pattern and the transcript group further comprises accounting for a probability of a given insert length for the RNA sequence read exhibiting the unique mutation pattern and transcript group. In some embodiments, calculation of the probability that the hypothetical DNA sequence from that haplotype will exhibit the unique mutation pattern further comprises accounting for a probability of a given insert length for the DNA sequence read exhibiting the unique mutation pattern.

In some embodiments, calculation of the probability that the hypothetical RNA sequence from that haplotype and transcript will exhibit the unique mutation pattern and the transcript group further comprises accounting for an RNA sequencing error probability. In some embodiments, calculation of the probability that the hypothetical DNA sequence from that haplotype and transcript will exhibit the unique mutation pattern and the transcript group further comprises accounting for a DNA sequencing error probability.

In some embodiments, estimating at least one of the set of haplotype-existence probabilities, the set of haplotype prevalences, and the set of haplotype-transcript prevalences comprises, for each haplotype of a set of possible haplotypes, using the statistical model to: sample a posterior probability distribution for haplotype existence to determine a point estimate for haplotype existence; sample a posterior probability distribution for haplotype prevalence to determine a point estimate for haplotype prevalence; and/or sample a posterior probability distribution for haplotype-transcript prevalence to determine a point estimate for haplotype-transcript prevalence. In some embodiments, the point estimate for each posterior probability distribution comprises a mean. In some embodiments, the posterior probability distribution for haplotype existence, haplotype prevalence, and/or haplotype-transcript prevalence is further used to determine an uncertainty associated with haplotype existence, haplotype prevalence, and/or haplotype-transcript prevalence, respectively.

In some embodiments, accessing the plurality of sequence reads further comprises accessing a set of germline variant and somatic mutation calls derived from tumor cells obtained from the subject.

In some embodiments, the statistical model comprises a probabilistic graphical model. In some embodiments, the statistical model comprises a hierarchical Bayesian model. In some embodiments, the hierarchical Bayesian model comprises a haplotype generation model, a DNA Dirichlet-Multinomial model, and/or an RNA Dirichlet-Multinomial model.

Also disclosed herein are systems including one or more computing devices, comprising: one or more non-transitory computer-readable storage media including instructions; and one or more processors coupled to the one or more storage media, the one or more processors configured to execute the instructions to perform any of the methods described herein.

Also disclosed herein are non-transitory computer-readable media comprising instructions that, when executed by one or more processors of one or more computing devices, cause the one or more processors to perform any of the methods described herein.

Disclosed herein are vaccines comprising: one or more peptides; a plurality of nucleic acids that encode the one or more peptides; or a plurality of cells expressing the one or more peptides, wherein the one or more peptides are selected from among a set of peptides by performing any of the methods described herein.

Disclosed herein are methods of manufacturing a vaccine comprising: performing any of the methods described herein to select one or more peptides from among a set of peptides; and producing a vaccine comprising: at least one of the one or more selected peptides; a plurality of nucleic acids that encode at least one of the one or more selected peptides; or a plurality of cells expressing at least one of the one or more peptides.

Disclosed herein are pharmaceutical compositions comprising one or more peptides selected from among a set of peptides by performing any of the methods described herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed can be resorted to by those skilled in the art, and that such modifications and variations are considered to be within the scope of this invention as defined by the appended claims.

### BRIEF DESCRIPTION OF THE DRAWINGS

FIGS. 1A-C illustrate various challenges of exemplary phasing analyses.

FIG. 1D illustrates the challenges in an exemplary read-backed phasing analysis with respect to DNA reads.

FIG. 1E illustrates the challenges in an exemplary read-backed phasing analysis with respect to RNA reads.

FIG. 2A illustrates an exemplary process for phasing mutations in a tumor of a patient, in accordance with some embodiments disclosed herein.

FIG. 2B illustrates an exemplary tumor mutation phasing system for performing one or more methods for phasing mutations in a tumor of a patient, in accordance with some embodiments disclosed herein.

FIG. 3 illustrates an exemplary sequence read counter for enumerating and quantifying unique mutation patterns observed in RNA and/or DNA sequence reads, in accordance with some embodiments disclosed herein.

FIG. 4 illustrates an exemplary sequence read counter for enumerating and quantifying unique mutation patterns and transcript groups observed in RNA sequence reads, in accordance with some embodiments disclosed herein.

FIGS. 5A-5B illustrate an exemplary enumeration and pattern estimator for determining a probability that a hypothetical DNA sequence read from a haplotype will exhibit a unique mutation pattern, in accordance with some embodiments disclosed herein.

FIG. 6A illustrates an exemplary enumeration and pattern estimator for determining a probability that a hypothetical RNA sequence from a haplotype will exhibit a combination of a unique mutation pattern and a transcript group, in accordance with some embodiments disclosed herein.

FIG. 6B provides a schematic illustration of the utility of determining haplotype-transcript prevalence when selecting tumor-associated peptides or proteins for the development of, e.g., personalized anti-cancer therapies.

FIG. 7 illustrates an exemplary diagram of a statistical model for phasing tumor haplotypes using RNA and/or DNA sequence data, in accordance with some embodiments disclosed herein.

FIG. 8A illustrates performance evaluation data from a simulation using techniques described herein, in accordance with some embodiments disclosed herein.

FIG. 8B illustrates performance evaluation data from a simulation using techniques described herein, in accordance with some embodiments disclosed herein.

FIG. 8C illustrates performance evaluation data for inferred values of φₕ (using the posterior mean as the estimator) that demonstrates close agreement with ground truth φₕ values in NA12878 genomic DNA.

FIG. 8D illustrates performance evaluation data for use of the disclosed methods to perform binary classification of haplotypes in NA12878 genomic DNA.

FIG. 8E illustrates performance evaluation data for use of the disclosed methods to perform binary classification of haplotypes in NA12878 genomic DNA.

FIG. 9 illustrates a flow diagram of a method for performing one or more methods for phasing mutations in a tumor of a subject, in accordance with some embodiments disclosed herein.

FIG. 10 illustrates an example computing system, in accordance with some embodiments disclosed herein.

### DESCRIPTION OF EXAMPLE EMBODIMENTS

### I. Overview

A phasing analysis restricted to normal tissue-reflecting a simpler problem than tumor phasing-involves separating maternally and paternally inherited copies of each chromosome into two haplotypes in order to get a clearer picture of genetic variation of a given subject. A haplotype refers to a set of genomic variants along a single chromosome that tend to be inherited together. The phasing analysis is needed because, in the sequencing data of a subject, the parental label of a given sequence read (i.e., whether the read comes from a maternal copy or paternal copy of a gene) is not directly observed, as illustrated below.

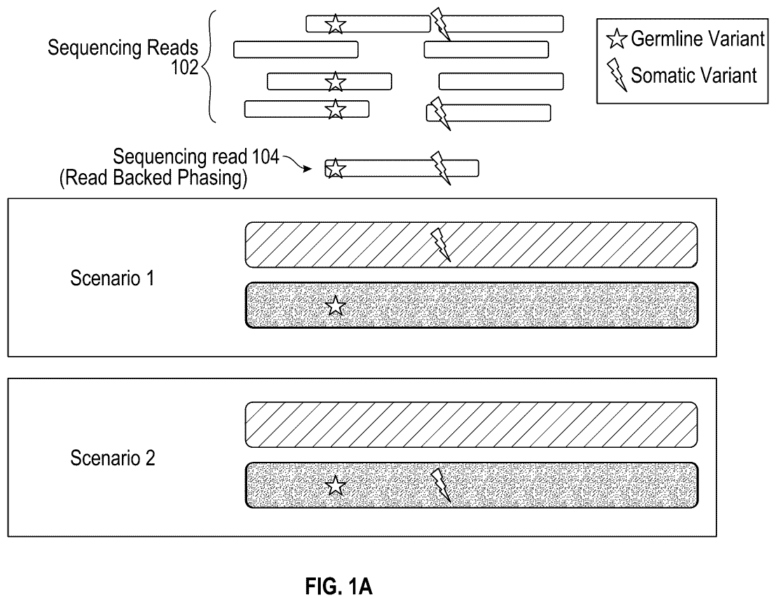

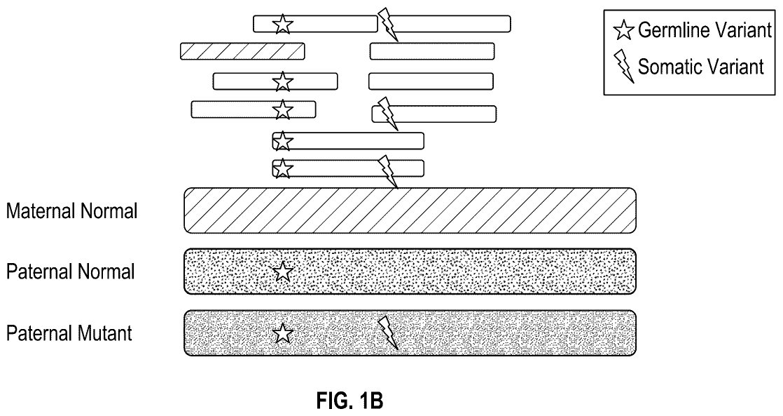

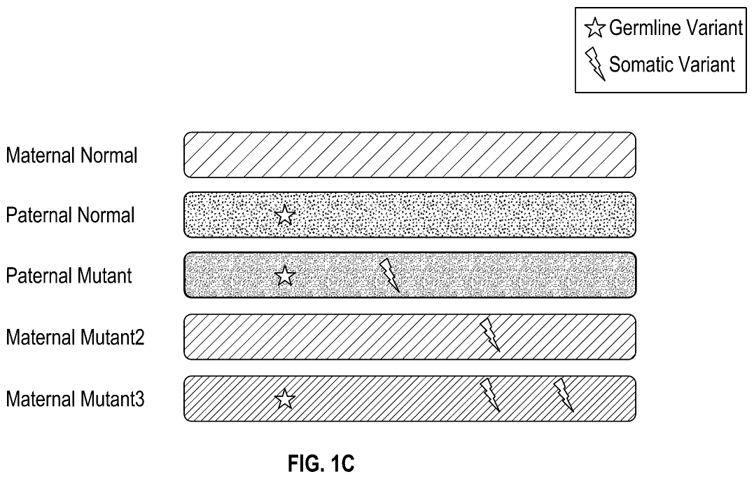

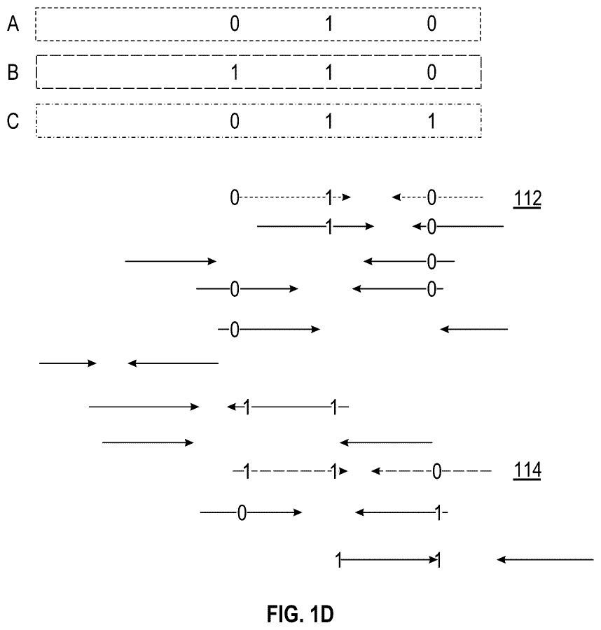

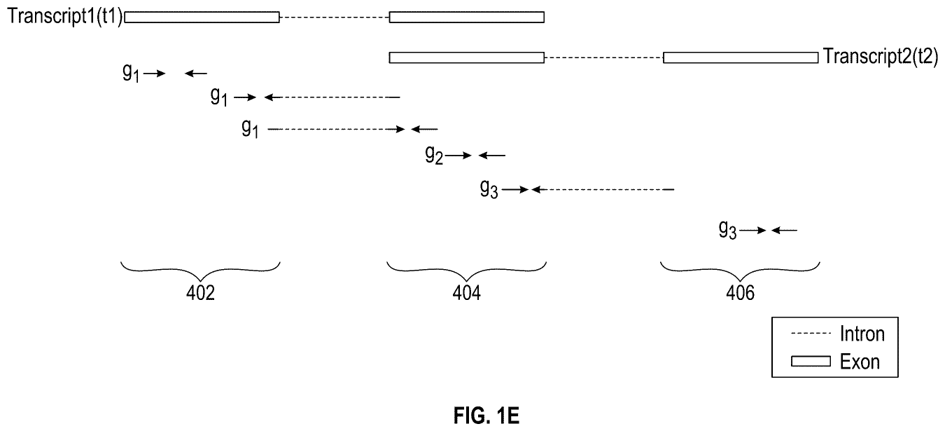

FIGS. 1A-C illustrate various challenges of exemplary phasing analyses. In FIG. 1A, a plurality of sequence reads 102 (e.g., DNA sequence reads) can be obtained from a subject, and some of the sequence reads contain genomic variants. However, the plurality of sequence reads 102 paints an ambiguous picture of the subject's genetic variation. For example, the reads 102 may occur due to scenario 1, which includes a maternal haplotype with a first genetic variant and a paternal haplotype with a second genetic variant. But alternatively, the reads 102 may also occur due to scenario 2, which includes a maternal normal haplotype and a paternal haplotype with both the first variant and the second variant. In the example in FIG. 1A, a hypothetical sequence read 104, which includes both the first genomic variant and the second genomic variant, can resolve the ambiguity because only scenario 2 can give rise to the sequence read 104. This is referred to as read-backed phasing, as described in more detail below.

FIG. 1B illustrates the tumor mutation phasing problem (with normal contamination), in accordance with some embodiments. During bulk sequencing of a tumor of a subject, both tumor cells and some normal cells of a subject are sequenced. The tumor cells contain somatic mutations and germline variants, while the normal cells may contain germline variants. Thus, as depicted in the example in FIG. 1B, bulk sequencing of tumors may result in 3 or more haplotypes generating reads, such as a maternal normal haplotype, a paternal normal haplotype (which contains a germline variant), and a paternal mutant haplotype (which contains both a germline variant and a somatic variant). As shown in FIG. 1B, the three haplotypes result in a complex mixture of sequence reads (e.g., DNA sequence reads), making it more challenging to discern the underlying haplotypes based on the sequence reads.

FIG. 1C illustrates the tumor mutation phasing problem with subclones, in accordance with some embodiments. FIG. 1C depicts five possible haplotypes of a subject, including a maternal normal haplotype, a paternal normal haplotype, a paternal mutant haplotype, a first maternal mutant haplotype, and a second maternal mutant haplotype. The five haplotypes again result in a complex mixture of sequence reads (e.g., DNA sequence reads), making it more challenging to discern the underlying haplotypes based on the sequence reads.

FIG. 1D illustrates the challenges in an exemplary read-backed phasing analysis with respect to DNA or RNA sequence reads. As shown in FIG. 1D, with multiple mutations, most sequence reads would not cover all mutations. This may be because the sequence reads are too short, the mutations occur within the paired end read insert region, and/or the aligned sequence reads are simply in a place where mutations do not occur. Furthermore, sequence reads may be affected by sequencing error. In the example in FIG. 1D, the sequence read 112, which indicates 0 at a first base position, 1 at a second base position, and 0 at a third base position (where 1 denotes the presence of an alternate allele and 0 denotes its absence) can be indicative of underlying haplotype A. The sequence read 114, which indicates 1 at the first base position, 1 at the second base position, and 0 at the third base position, can be indicative of underlying haplotype B. However, the majority of the reads do not definitively indicate the presence of underlying haplotypes due to the problems described above.

FIG. 1E illustrates an exemplary read-backed phasing analysis with respect to RNA sequence reads. An RNA transcript is a single-stranded ribonucleic acid molecule synthesized from a DNA template during transcription. Some of these RNA transcripts can encode proteins, and prior to processing, these are known as precursor mRNA molecules. As part of the process of converting precursor mRNA into mature mRNA, regions known as ‘introns’ are spliced out by a spliceosome molecule, after which regions known as ‘exons’ remain; some of these exonic regions will encode an eventual protein product following translation. Crucially, precursor mRNA can be spliced in multiple (but finite) ways, with some transcripts retaining one subset of exons, and other transcripts retaining a different subset of exons; and regions that are intronic in one transcript may be exonic in another. Each subset of exons that is known to possibly remain after splicing is referred to as a transcript isoform. On average, each protein coding gene is associated with ˜4 known transcript isoforms. Also, transcript isoforms for a given gene usually share exons with one another. Finally, note that although ‘transcript’ is typically a term used to describe individual RNA molecules—not the patterns of exons that can remain following splicing, which are most accurately referred to as “transcript isoforms”—transcript isoforms will be referred to simply as transcripts in this document. References to transcript molecules will include explicit use of the word ‘molecule’.

In some embodiments, mature mRNA sequence read data is used to perform the techniques described herein to improve mutation phasing. Mature mRNA sequencing detects and quantifies the expressions of transcript sequences that have already had introns spliced out. This is in contrast with total RNA sequencing, which can detect and quantify the expression of immature transcripts. Mature mRNA data can be more relevant as it phases only those mutations that are not spliced out and accordingly might eventually be translated into a peptide; i.e., a potential neoantigen target. It should be appreciated that techniques described herein can also be used to phase mutations found in total RNA sequencing, for example, by using different immature transcript models.

FIG. 1E illustrates two exemplary RNA transcripts. As shown in FIG. 1E, each of RNA transcript t1 and RNA transcript t2 includes two exons connected with an intron. One of the exons is shared between both transcripts.

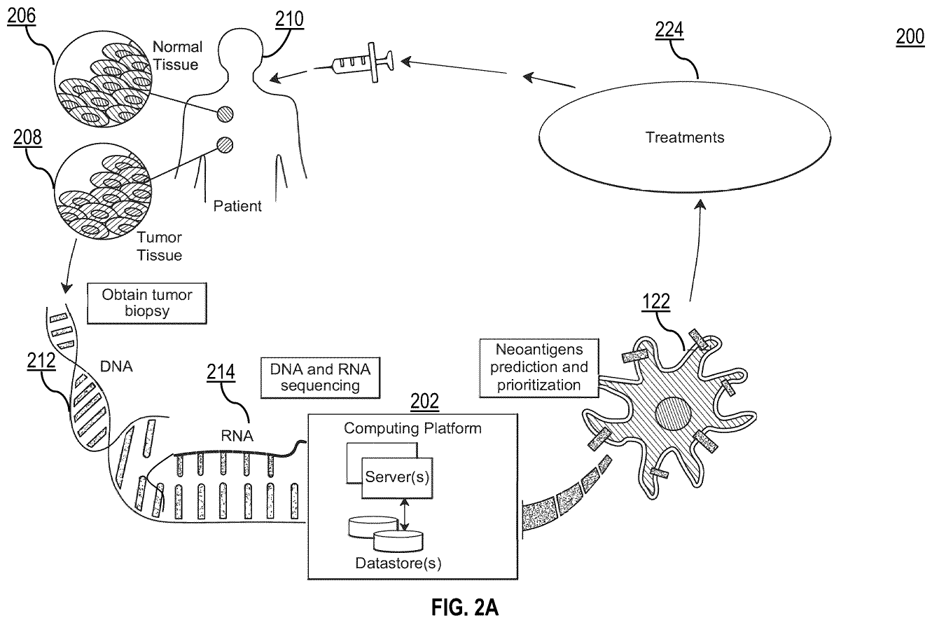

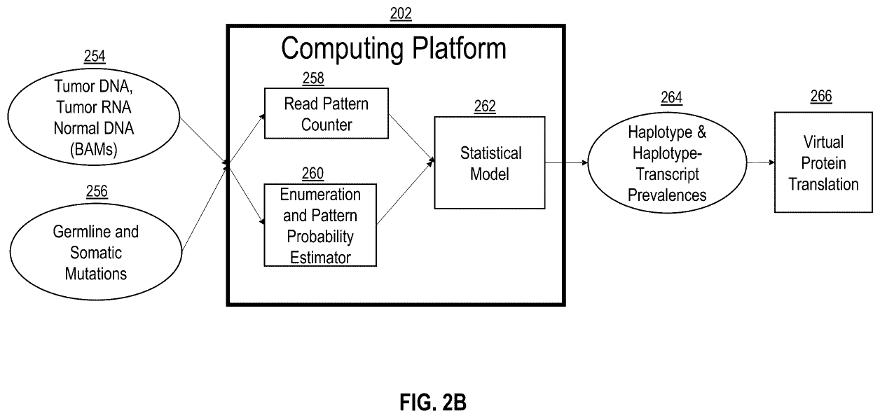

For a given gene, multiple disjoint transcript “regions” can be defined. Each region is also described as being consistent with a specific ‘transcript group’, where each transcript group is one of the subsets of the transcripts expressed by a gene. A region is defined as being consistent with a transcript group if every base within that region is in part of an exon that is expressed by every transcript in that transcript group—and that is not expressed in any transcript outside the transcript group—such that every position within an exon in a gene can be classified as being part of a single unique transcript group. Generally, for a given gene, a system (e.g., Enumeration and Pattern Probability Estimator 260 illustrated in FIG. 2B) can divide it into a region exclusive to transcript t1, another exclusive to transcript t2, . . . , another exclusive to transcript tj, and for all pairs j and k, into disjoint regions shared only by tj and tk, for all j!=k, and repeat for all triplets (j,k,l), and so on, until the entirety of the gene sequence (i.e., every base position within every exon in the gene) is classified as belonging to one of these disjoint regions. In other words, transcript groups can be defined for all singletons (transcript groups comprising a single transcript), pairs (transcript groups comprising a pair of transcripts), triplets (transcript groups comprising three transcripts), quads (transcript groups comprising four transcripts), . . . , etc. Of note, a single transcript can give rise to reads belonging to multiple transcript groups; some exons within the transcript are shared amongst one set of transcripts, whereas other exons within the transcript are shared amongst a different set of transcripts.

In the depicted example in FIG. 1E, for two transcripts, there is one region 402 exclusive to transcript t1, a second region 406 exclusive to transcript t2, and a third region 404 shared by t1 and t2. As another example, if there are three transcripts (t1, t2, t3), there would be seven transcript groups corresponding to (t1), (t2), (t3), (t1, t2), (t1, t3), (t2, t3), and (t1, t2, and t3).

Potential transcript groups can be enumerated by taking the combination of all subsets—excluding the empty subset—of all possible transcripts overlapping a given gene or gene region. So, if a given gene or gene region corresponds to 3 transcripts as described in the previous example (where the number of transcripts is derived from, e.g., an existing gene transfer format GTF) file) as described in the previous example, there are N=(2³−1) transcript groups (i.e., again corresponding to (t1), (t2), (t3), (t1, t2), (t1, t3), (t2, t3), and (t1, t2, t3) in this case). Once the set of transcript groups has been enumerated, a given sequence read can be assigned to a specific transcript group by performing steps comprising, for example: (i) identifying, for each base within a sequence read, one or more transcripts that have that base at that genomic coordinate as indicated in the aforementioned GTF file (or no transcripts may be identified for the sequence read if there are no transcripts that have that base at that genomic coordinate), (ii) if a base is part of more than one transcript in the GTF, it is assigned to the transcript group that includes each of those transcripts—and that includes no other transcript, and (iii) the overall sequence read is assigned to the transcript group that is the smallest of the transcript groups for any base within that sequence read. As an example: assume that: 80% of a sequence read comprises bases which could be assigned to transcript 1, transcript 2, or transcript 3; assume that 5% of the sequence read comprises a base which could be part of transcript 1 or transcript 2; and assume that 15% of the sequence read comprises a base which could be assigned only to transcript 1. In this case, as transcript 1 by itself is the smallest transcript group, the sequence read is assigned to transcript group 1.

Read-backed phasing can be performed to assign each RNA sequence read to a single unique transcript group, but such transcript group assignments (or labels) are, by construction, ambiguous with respect to assignment of RNA sequence reads to a specific single transcript. In the example in FIG. 1E, each of the reads labeled g1 can only come from transcript t1 because it aligns at least partially to an exon in region 402 that is unique to transcript t1. Similarly, each of the reads labeled g3 can only come from transcript t2 because it aligns at least partially to an exon in region 406 that is unique to transcript t2. However, the read labeled g2 aligns with region 404 that is common to both transcript t1 and transcript t2, thus leading to ambiguity that phasing analysis needs to account for.

Disclosed herein are exemplary devices, apparatuses, systems, methods, and non-transitory storage media for phasing mutations in a tumor of a subject. An exemplary system can access sequence reads derived from tumor cells obtained from the subject. The sequence reads can comprise tumor DNA sequence reads and/or tumor RNA sequence reads. In accordance with the techniques described herein, the system can analyze the sequence reads using a novel statistical model for tumor mutation phasing to estimate, for one or more haplotypes, one or more of a set of probabilities that each of the haplotypes exists (also referred to as haplotype-existence probabilities), a set of haplotype prevalences, and a set of haplotype-transcript prevalences (the prevalence of molecules from a specific haplotype and simultaneously encoded by specific transcript).

In one example, for haplotype A, the system can estimate a posterior probability distribution on the event that haplotype A exists, on the prevalence of haplotype A, and/or on one or more haplotype-transcript prevalence values associated with haplotype A. These posterior probability distribution estimates can in principle be used in their raw sample-based form in subsequent analyses (among other benefits, potentially informing subsequent steps of the degree of uncertainty), or they can be summarized (via mean, median or some other statistic) into point estimates of (i) the probability that haplotype A exists, and/or (ii) the haplotype prevalence of A, and/or, (iii) if RNA sequence data is available, the prevalence of haplotype A as expressed in each transcript, relative to the set of all RNA transcript molecules. Accordingly, embodiments of the present disclosure can analyze DNA and/or RNA sequence reads derived from tumor cells obtained from the subject, impute (by matching sequence reads to unique mutation patterns identified in the sequence read data) what underlying haplotypes are represented in the sequence read data, and perform a statistical analysis (e.g., using a statistical model) to generate a set of posterior probability distributions for i) the existence of each haplotype, and/or ii) the prevalence of each haplotype, and/or iii) the prevalence of each haplotype-transcript combination. In some instances, one or more of these posterior probability distributions can be used directly in downstream analyses. In some instances, these posterior probability distributions can be used to calculate a summary statistic (e.g., a mean, median, or other statistic) as a point estimate for use in downstream analyses.

Accordingly, embodiments of the present disclosure can model sequence data derived from a tumor sample, which can include normal cells, polyclonal tumor cells, copy number alterations, and other features that may break the assumptions of other phasing analysis tools described above. Embodiments of the present disclosure can exploit tumor-normal-RNA-seq (T-N-R) triplet data from the same patient and simultaneously model DNA and RNA. Further, embodiments of the present disclosure can jointly model haplotype prevalences and haplotype-transcript-group prevalences.

Further, embodiments of the present disclosure can estimate the number of haplotypes present in a bulk sample (e.g., the number of haplotypes per gene) and do not assume that the number of haplotypes is known. Existing methods may operate under the assumption that a fixed number of haplotypes exist in the sample. However, in reality there can be almost any number of haplotypes in the sample. Embodiments of the present disclosure do not assume a fixed number of haplotypes or operate under that constraint. In fact, embodiments of the present disclosure can be used to estimate the number of haplotypes present in the sample (e.g., by estimating the probability that each haplotype of a plurality of possible haplotypes exists). In some embodiments, the system can output a list of haplotypes present in the sample (e.g., a list of haplotypes associated with existence probabilities above a predefined threshold).

The exemplary system can quantify parameter uncertainty, incorporate prior information about likely haplotypes, and estimate transcript-specific haplotype prevalences. In some instances, embodiments of the present disclosure can estimate the posterior probability distribution of haplotype prevalence and the posterior probability distribution of haplotype-transcript prevalence, rather than determining point estimates for these parameters.

Embodiments of the present disclosure can be used to accurately model proteins and develop patient-specific cancer treatments. For example, embodiments of the present disclosure can be used in a pipeline for characterizing and ranking candidate neoantigens for use in vaccine development. As discussed below, the pipeline can use a combination of tumor DNA sequencing data, normal DNA sequencing data (e.g., from peripheral blood mononuclear cells (PBMC) or tumor-adjacent tissue), and/or tumor RNA sequencing data, where the reads may be short or long, DNA sequencing may be WES, WGS, sWGS, tumor targeted sequencing, or any other similar DNA sequencing approach, and tumor RNA sequencing may be ribosomally depleted RNA sequencing, poly-A mRNA sequencing, amplicon sequencing, hybridization capture sequencing, total RNA-sequencing, or any other similar RNA sequencing approach.

Embodiments of the present disclosure can be used to perform the phasing analysis to accurately estimate the presence and prevalence of various haplotypes, which are then used to identify mutant proteins (neoantigens) in the tumor. For example, a set of haplotypes identified in the sequence read data (e.g., based on comparison of haplotype-existence probabilities to a predefined threshold) can be translated in silico to determine tumor-associated peptide or protein sequences (e.g., by parsing nucleotide sequence data into codons, and then translating the codons into a corresponding amino acid sequence). Haplotype prevalence data output by the statistical model can be used to rank-order and/or select a subset of the tumor-associated peptide or protein sequences for use in developing patient-specific therapies (e.g., anti-cancer therapies). Haplotype-transcript prevalence data output by the statistical model can be used to further refine the rank-ordering and/or selection of the subset of the tumor-associated peptide or protein sequences for use in developing patient-specific therapies (e.g., anti-cancer therapies), for example, by determining that the transcript for one tumor-associated peptide or protein is much more abundant than that for a different tumor-associated peptide or protein.

The pipeline can be used to characterize and select the patient-specific neoantigens (e.g., based on the predicted immunogenic properties of the proteins) to be targeted by vaccines. In some embodiments, the pipeline can identify and/or select a predefined number of neoantigens for each patient (e.g., 5, 10, 20, or more than 20 neoantigens may be selected for each patient), and identify individualized neoantigen-specific therapies to induce immune response in a given patient. In some embodiments, the neoantigen-specific therapies can induce priming by RNA vaccination, DNA vaccination, infusion of engineered and primed T-cells, or any combination thereof.

Embodiments of the present disclosure can be valuable in any use cases involving the modeling of tumor proteins. Depending on indication, it is estimated that up to 10% of somatic missense mutations are near in-phase mutations and are currently ignored without the phasing analysis. Further, previously published results suggest the lack of proper phasing analysis may lead to a 7% false positive rate of predicting p-MHC binding, and predicted binding affinity could be reduced 70-fold by the presence of one in-phase and proximal germline variant. Further, priming for the wrong peptide (e.g., a non-existent peptide) could prime ineffective T cells in the context of antigen-focused immunotherapy. In general, and more broadly, embodiments of the present disclosure can be useful anytime a tumor protein needs to be accurately modeled, since proteins arising from different haplotypes may have different properties, different structures, and different immunogenic qualities.

In some embodiments, in order to estimate the probabilities and prevalence values described above, the exemplary system can determine a number of inputs for a statistical model based on the sequence reads, and provide the inputs to the statistical model. For example, the exemplary system can identify and enumerate a set of unique mutation patterns (i.e., all possible combinations of observed variant alleles that could potentially be revealed by a read aligning within a gene or gene region) for the set of mutations that are input to the system. a. A mutation pattern indicates a specific combination of variant alleles. One exemplary unique mutation pattern may be “1, 0, 1”, which indicates an allele value “1” at a first base position, an allele value “0” at a second base position, and an allele value “1” at a third base position, where 1 denotes the presence of an alternate allele and 0 denotes the presence of the wild type allele. Another exemplary unique mutation pattern may be “1, 1, 1”, which indicates an allele value “1” at the first base position, an allele value “1” at the second base position, and an allele value “1” at the third base position. For each identified unique mutation pattern, the system can further count how many times the unique mutation pattern has actually been observed in sequence reads. For example, the system can count that the unique mutation pattern “1, 1, 1” has appeared in two sequence reads, and thus determines that the quantity of the mutation pattern “1, 1, 1” to be 2. In some embodiments, a mutation pattern may comprise a “?” at a given base position (i.e., the allele is not observed by a particular read at the given base position) because many sequence reads in short read phasing may not cover all of the mutations present in a given gene. Accordingly, the system can enumerate one or more unique mutation patterns and, for each unique mutation pattern, determine an associated mutation pattern quantity (e.g., by counting the number of sequence reads that exhibit a given unique mutation pattern).

As a second input to be provided to the statistical model, the exemplary system can determine, for each unique mutation pattern, a probability, for each haplotype of a set of candidate haplotypes, that a sequence read from the specific haplotype will exhibit the specific mutation pattern. Such a probability can be determined, e.g., based on the haplotype variant positions, on the specified length of the read and on its relative position with respect to the candidate haplotype sequence (for example, by enumerating all possible sequence reads that could be generated for the haplotype sequence, and that are consistent with the mutation pattern, by shifting the ends of a sequencing read of a specified length along the haplotype sequence one base at a time, performing a weighted summation of the number of these sequence reads that would exhibit the specific mutation pattern, where each weight is the probability of that read pair's insert length, multiplied by the probability of the required amount of sequencing error needed for the candidate haplotype to generate that sequence read, and dividing this weighted sum by the total weighted sum of all possible sequence reads—irrespective of mutation pattern—that could be generated by the specific haplotype sequence. For example, the system can determine a first probability indicative of how likely a hypothetical DNA sequence read from the haplotype A will exhibit unique mutation pattern “1, 0, 1.” Further, the system can determine a second probability indicative of how likely a hypothetical DNA sequence read from the haplotype A will exhibit the unique mutation pattern “1, 1, 1.” Further, the system can determine a third probability indicative of how likely a hypothetical DNA sequence read from the haplotype A will exhibit the unique mutation pattern “1, 1, ?”, where “?” indicates a mutation position that is not covered by a given individual read such that the allele that position is not determined. Accordingly, a plurality of probabilities can be determined for a given haplotype corresponding to a plurality of unique mutation patterns.

As a third input to be provided to the statistical model, the exemplary system can enumerate combinations of the set of unique mutation patterns and the set of unique transcript groups potentially observed in RNA sequence reads. As a fourth input to be provided to the statistical model, the exemplary system can determine, for each combination of a haplotype from a set of candidate haplotypes and transcripts for a given gene, a a probability that a hypothetical RNA sequence read from that haplotype and transcript will exhibit a given unique mutation pattern and unique transcript group. For example, the system can determine, for a combination of haplotype A and a first transcript, a probability that a hypothetical RNA sequence read from that haplotype will exhibit the unique mutation pattern “1, 0, 1” in the given combination of haplotype A and the first transcript group, where the probability is determined in a manner analogous to that described above for DNA sequence reads—the primary difference being that counting is performed conditional on the specific haplotype and the specific transcript. As another example, the system can determine, for a combination of haplotype A and a second transcript, a probability that a hypothetical RNA sequence read from that haplotype will exhibit the unique mutation pattern “1, 0, 1” in the given combination of haplotype A and some other second transcript group.

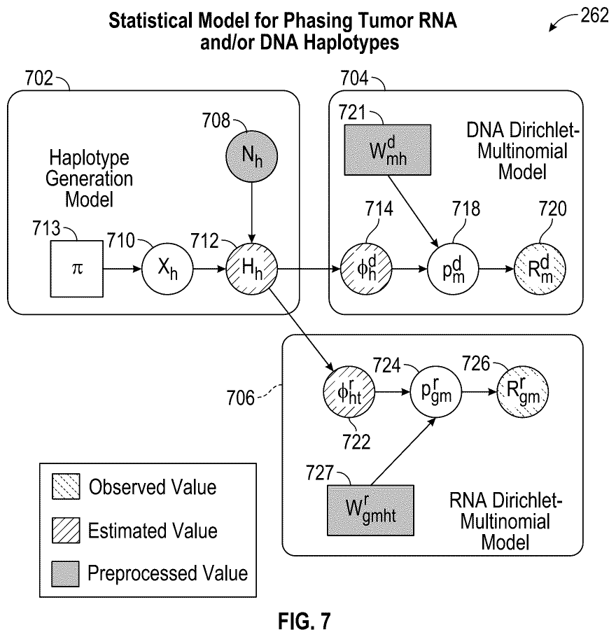

The exemplary system can input the mutation pattern quantities and the mutation pattern probabilities into the statistical model to estimate one or more of a set of i) probabilities that each of a set of haplotypes exists (also referred to as haplotype-existence probabilities), ii) a set of haplotype prevalences, and/or iii) a set of haplotype-transcript prevalences. In some embodiments, the statistical model comprises a hierarchical Bayesian model (i.e., a statistical model written in multiple levels (hierarchical form) that estimates the parameters of a posterior probability distribution using the Bayesian inference method to update probabilities as additional data is provided). As illustrated in FIG. 7 and described below, the hierarchical Bayesian model may include several components, e.g., a haplotype generation model used to determine what haplotypes are present in the sequence read data, a DNA Dirichlet-Multinomial model used to determine haplotype prevalence from DNA sequence read data, and/or an RNA Dirichlet-Multinomial model used to determine haplotype-transcript prevalences from RNA sequence read data. It should be appreciated by one of ordinary skill in the art that other statistical models may be used to perform the techniques described herein.

### Model Description

Additional description of an exemplary model, including alternative statistical models that could be used, is provided below.

### Phasing-Window Model

The phasing window-specific model structure (as opposed to models for am and p(l), discussed below, which, as preprocesses for the phasing window-specific model, simultaneously account for sequence reads from across the genome) allows for the integration of a haplotype sampling model, a DNA sampling model, and an RNA sampling model.

### Haplotype Model

Haplotypes can be modelled as follows:

$$π \sim Beta ( 1 , 1 )$$

$${X}_{h} \sim Ber n ( π ) {H}_{h} = {N}_{h} + {X}_{h} - {N}_{h} \cdot {X}_{h}$$

where:
Hₕ is a binary variable denoting whether haplotype h occurs in the tissue sequenced from the tumor sample
Xₕ is a binary variable denoting whether haplotype h is actually in the tumor (as opposed to the tumor sample, which may be contaminated)
Nₕ is a binary variable indicating whether haplotype h is known to be one of the normal haplotypes. Nₕ could be determined by using an off-the-shelf germline variant phasing method, or by applying the DNA-only model to normal data, as though that data were from the tumor. Of note, when Nₕ is 1, Hₕ is by definition 1 as well, in order to reflect normal contamination, but this could be loosened to be a probabilistic relationship.

### RNA Model

RNA reads arising from a given haplotype can be modelled as follows (effectively, a complicated version of a dirichlet-multinomial, in which each haplotype can generate more than one type of read, and each type of read can arise from more than one kind of haplotype):

$${ϕ}_{ht}^{r} ∼ Dirichlet ( {λ}_{ht} = {α}_{ht} + {H}_{ht} )$$

$${α}_{h t} = \text{.001}$$

$$\begin{matrix}{p}_{g m} = \frac{{\sum }_{h t l j a} I ( h , t , l , j , g , a ) \times {δ}_{am}^{r} \times {ϕ}_{h t}^{r} \times p ( {l}^{r} )}{{\sum }_{{htljag}^{′} {m}^{′}} I ( h , t , l , j , {g}^{′} , a ) \times {δ}_{{am}^{′}}^{r} \times {ϕ}_{h t}^{r} \times p ( {l}^{r} )} \\ = \frac{{\sum }_{h t} {W}_{g m h t} \times {ϕ}_{h t}^{r}}{{\sum }_{htg {′}^{′} {m}^{′}} {W}_{gmht} \times {ϕ}_{h t}^{r}}\end{matrix}$$

$${R}_{g m}^{r} ∼ Multinomia1 ( {R}^{r} , {p}_{g m} )$$

Where:

$${ϕ}_{h t}^{r}$$

is the prevalence of RNA molecules of haplotype h and transcript t
λₕₜ is the dirichlet parameter for sampling

$${ϕ}_{h t}^{r} .$$

This form of dirichlet prior, which we can refer to as a ‘spike-and-slab’ prior, assigns λₕₜ to be a value slightly larger than 1 for haplotypes that are present (slab), and assigns λₕₜ to be αₕₜ, a very small value, for haplotypes that are not present (spike). The primary purpose of this very small αₕₜ term was to ensure that the dirichlet was a proper prior for

$${ϕ}_{h t}^{r}$$

—it prevents any of the λ from being exactly 0. But it can also be thought of as a catch all to account for any remaining error that isn't modelled explicitly by the sequencing error probability, δₐₘ
αₕₜ is the very small constant for the dirichlet, which we typically simply set to 0.001. An alternative might be to sample αₕₜ from some gamma having expectation close to 0; we found this choice didn't affect results.
p_{gm} is the total probability, accounting for the possibility of any pairs of haplotypes h and transcript t, of generating an RNA read having mutation pattern m, and exhibiting transcript group g.
I(h, t, g, l, a) is an indicator denoting whether a read from haplotype h and transcript t, starting at position j, with insert length l, and actual allele a, will exhibit mutation pattern m and transcript group g

$${δ}_{a m}^{r}$$

is the probability that a RNA read having actual mutation pattern a will be sequenced as though it were mutation pattern m. This parameter is determined by fitting a frequentist Bernoulli model to RNA reads from across the entire genome. For a=m, the value will be close to 1, and for a≠m, the value will be close to 0, depending on the likelihood of sequencing error.
p^{r}(l) is the probability that an RNA read pair has insert length l. This parameter is determined by fitting a frequentist negative binomial model to those RNA reads from across the entire genome that are in long, single-transcript exons.
W_{gmht} is the non-normalized probability that a RNA read from haplotype h and transcript t will exhibit mutation pattern m and transcript group g.

$${R}_{g m}^{r}$$

is the number of RNA reads from within the phasing window that exhibit mutation pattern m and transcript group g.
R^{r} is the total number of RNA reads in the phasing window

### DNA Model

DNA reads can be modelled similarly, i.e., they can be treated effectively as though they are RNA reads arising from a gene with a single transcript, so the equations no longer include indexes per transcript or transcript group.

$${ϕ}_{h}^{d} ∼ Dirichlet ( {λ}_{h} = {α}_{h} + {H}_{h} )$$

$${α}_{h} = . 001$$

$$\begin{matrix}{p}_{m} = \frac{{\sum }_{h l j a} I ( h , l , j , a ) \times {δ}_{am}^{d} \times {ϕ}_{h}^{d} \times p ( {l}^{d} )}{{\sum }_{h l {jam}^{′}} I ( h , l , j , a ) \times {δ}_{{am}^{′}} \times {ϕ}_{h}^{d} \times p ( {l}^{d} )} \\ = \frac{{\sum }_{h} {W}_{m h} \times {ϕ}_{h}^{d}}{{\sum }_{{hm}^{′}} {W}_{{m}^{′} h} \times {ϕ}_{h}^{d}}\end{matrix}$$

$${R}_{m}^{d} ∼ Multinomial ( {R}^{d} , {p}_{m} )$$

$${ϕ}_{h}^{d}$$

is the prevalence of DNA molecules of haplotype h
λₕ is the dirichlet parameter for sampling

$${ϕ}_{h}^{d} .$$

This is also a dirichlet ‘spike-and-slab’ prior.
αₕ is the very small constant for the dirichlet, which we typically simply set to 0.001.
pₘ is the total probability, accounting for all possible haplotypes h of a given DNA read having mutation pattern m.
I(h, l, j, a) is an indicator denoting whether a DNA read from haplotype h starting at position j, with insert length l, and actual allele a, will exhibit mutation pattern m.

$${δ}_{a m}^{d}$$

is the probability that a DNA read having actual mutation pattern a will be sequenced as though it were mutation pattern m.
p^{d}(l) is the probability that an DNA read pair has insert length l. As described earlier, this parameter is determined by fitting a frequentist negative binomial model to DNA reads from across the entire genome.
Wₘₕ is the non-normalized probability that a DNA read from haplotype h will exhibit mutation pattern m.

$${R}_{m}^{d}$$

is the number of DNA reads from within the phasing window that exhibit mutation pattern m.
R^{d} the total number of DNA reads in the phasing window

### Alternative Formulations and Solutions

There are many potential alternative variations of this model. Some of these have been mentioned already. These alternative variations can include, but are not limited to:
Specifying

$${ϕ}_{h}^{d}$$

(rather than Hₕ) as an explicit parameter of the prior for

$${ϕ}_{h t}^{r} ,$$

encoding the assumption that haplotype-transcript specific expression is correlated with haplotype prevalence.
Weighting the prior for sampling each Xₕ according to how similar the Xₕ germline variant phasing structure is to a known-to-exist normal haplotype Nₕ; this encodes the assumption that tumor haplotypes might tend to preserve germline variant structure.
Suppose the number of haplotypes P in a phasing window is already known (e.g., from a preprocessing method that estimates clonality). Enforcing a constraint on the number of haplotypes can inform the prior on haplotype existence. Specifically, to determine Xₕ, the model could sample unique h values without replacement from the set of all possible haplotypes 1: [symbol] , until P such unique h values are sampled, and assign Xₕ=1 for all h values that are sampled.
   Elaborating on the idea of a model that is parameterized by a fixed number of haplotypes P: if the parameter P is not known, the P parameterized model can be repeatedly fit for all values P∈1:maxP, and then the model with the ‘best’ fit can be selected
Specifying a hyperprior for αₕ and αₕₜ rather than a fixed hyperparameter.
Removing the variables Hₕ and Nₕ from the model altogether and simply sample

$${ϕ}_{h}^{d}$$

and/or

$${ϕ}_{h t}^{r}$$

from an uninformative Dirichlet. This would likely result in a less parsimonious mole fit.
The ordering of Nₕ and Hₕ in the model could be reversed, with Hₕ informing the prior for Nₕ, and Nₕ being a parameter that is simultaneously fit along with all other unknown variables in the model, rather than being a preprocessed known value.

Alternative approaches to JAGS MCMC sampling are also available for fitting this phasing model or a similar one; including but not limited to:
STAN's Hamiltonian Monte-Carlo sampler.
An expectation-maximization approach for determining maximum likelihood parameter estimates.
Approximating the posterior of the original model as a function of, e.g., multivariate normal random variables, and fitting it via Variational Bayes.

### II. Platform Overview

FIG. 2A illustrates an exemplary process 200 for phasing mutations in a tumor of a patient, in accordance with the presently disclosed embodiments. In certain embodiments, the workflow system 200 may include a combination of a laboratory workflow and a computing workflow. With reference to FIG. 2A, as a part of the laboratory workflow, a biopsy of one or more samples can be performed on a patient 210. The one or more samples can include a healthy sample from healthy tissue, a tumor sample from tumorous tissue, or a combination thereof. Further, a tumor sample can include normal (i.e., healthy) tissue or cells 206, cancer tissue or cells 208, or a combination thereof.

The laboratory workflow may further include determination of DNA sequences 212 and RNA sequences 214 from the one or more samples. For example, a tumor sample from the patient 210 may be obtained, and DNA and/or RNA may be isolated from the tumor sample using any method known in the art. DNA and/or RNA sequences can also be determined by any sequencing method known in the art. In some embodiments, next-generation sequencing methods involving massive parallel sequencing are utilized to obtain short-read sequences, which are typically between 50 and 400 bases in length. In some embodiments, the DNA template to be sequenced may be obtained by clonal emulsion PCR or clonal bridge amplification. In certain embodiments, the laboratory workflow may include a hybrid capture subprocess, in which one or more sequence reads are generated based on sequencing of the DNA and/or RNA extracted from the one or more samples. “Hybridization capture” refers to a targeted next generation sequencing method that involves enrichment of a target genomic sequence through hybridization of a tagged (e.g., biotinylated) bait oligonucleotide to a region of interest on a DNA fragment, and capture of the bait oligonucleotide via the tag (e.g., using streptavidin-conjugated magnetic beads) before sequencing. In some embodiments, third generation sequencing methods can be utilized to obtain long-read sequences, which are typically greater than 10-1000 kilobases in length. In certain embodiments, third generation sequencing methods developed by Oxford Nanopore Technology can be utilized. In other embodiments, third generation sequencing methods developed by Pacific Biosciences involving single molecular real time sequencing can be employed. In certain embodiments, the one or more sequence reads generated by sequencing the DNA and/or RNA extracted from the one or more samples can include tumor DNA sequence reads, tumor RNA sequence reads, normal DNA sequence reads, or a combination thereof.

In one embodiment, the sequencing subprocess may generate raw base call data as a Binary Base Call (BCL) file, which may be then converted and mapped into one or more sorted binary alignment map (BAM) files. For example, each sorted BAM file may include the data for one or more sequence reads (e.g., tumor DNA sequence reads, tumor RNA sequence reads, and normal DNA sequence reads) generated by sequencing the DNA and/or RNA extracted from the one or more samples. In certain embodiments, a computing platform 202 can receive the sorted BAM files including the base call data for the sequence reads (e.g., tumor DNA sequence reads, tumor RNA sequence reads, and normal DNA sequence reads) and further perform one or more genomic variant calls (e.g., germline variant calls, somatic variant calls) utilizing the sorted BAM files of sequence read data to identify germline and/or somatic mutations.

In certain embodiments, as further depicted by FIG. 2A, the computing platform 202 may include one or more computing devices (e.g., one or more servers and/or client devices) and one or more databases (e.g., data stores, relational databases). For example, in some embodiments, the computing platform 202 may include a cloud-based computing architecture suitable for performing techniques for phasing mutations in a tumor of a patient 210 in accordance with the presently disclosed embodiments. For example, in one embodiment, the computing platform 202 may include a Platform as a Service (PaaS) architecture, a Software as a Service (SaaS) architecture, an Infrastructure as a Service (IaaS) architecture, a Compute as a Service (CaaS) architecture, a Data as a Service (DaaS) architecture, a Database as a Service (DbaaS) architecture, or other similar cloud-based computing architecture (e.g., “X” as a Service (XaaS)).

The computing platform 202 may perform techniques for phasing mutations in tumors of subjects as described herein. In certain embodiments, the computing platform 202 may utilize a statistical model (e.g., a hierarchical Bayesian model) to estimate one or more of a set of probabilities that each of a number of haplotypes exists in a sample (also referred to as haplotype-existence probabilities), as evidenced by the sequencing reads from that sample (e.g., tumor DNA sequence reads, tumor RNA sequence reads, and/or normal DNA sequence reads), a set of haplotype prevalences (from DNA and/or RNA sequence read data), and a set of haplotype-transcript prevalences (from RNA sequence read data) based on the sequence reads and the identified germline and/or somatic mutations, as will be further described with respect to FIG. 2B below. In some embodiments, tumor DNA sequence reads and normal DNA sequence reads may be aligned to a reference genome (using, e.g., the BWA and STAR aligners respectively) and compared to call somatic mutations (e.g., using the MuTect or VarSim callers).

In certain embodiments, as further depicted by FIG. 2A, the exemplary system can determine one or more individualized cancer immunotherapy treatments 224 based on the phasing of mutations in the sequence read data. For example, in some embodiments, the computing platform 202 may generate a prediction of tumor neoantigens, which may include a set of mutant peptide sequences derived from expressed somatic mutations in the sequence read data associated with the patient 210. Specifically, in some embodiments, the phased mutations may be in silico translated into the set of tumor neoantigens (e.g., mutant peptide sequences). In some embodiments, the system can determine which haplotypes are present based on the haplotype-existence probabilities (e.g., by determining if the probability is non-zero or exceeds a predefined threshold). Those haplotypes that are present can be translated to peptide sequences without the use of the prevalences. In other words, the system can limit the downstream analysis on those haplotypes that are known to be present based on the sequence read data so as to avoid targeting a haplotype that is not present since it would not be effective as a treatment. The estimated prevalences (e.g., haplotype prevalences and/or haplotype-transcript prevalences) are used to select one or more corresponding peptide sequences (e.g., 1, 2, 3, 4, 5, 6, 7, 8, 9, 10, or more than 10 corresponding peptide sequences) to target in a vaccine or for use in a pharmaceutical composition. For example, if the prevalence estimate for a given haplotype is greater than a threshold value, the corresponding peptide sequence can be selected for further downstream processing. On the other hand, if the prevalence estimate for a given haplotype is lower than the threshold value (e.g., indicative of very low prevalence), the system can exclude the haplotype (and its corresponding peptide sequence) from downstream processing so as to avoid targeting a neoantigen associated with a haplotype with a low prevalence in a vaccine. Alternatively, peptide sequences can be ranked at least in part according to their associated haplotype-transcript prevalences, and some subset of those peptide sequences (e.g., 1, 2, 3, 4, 5, 6, 7, 8, 9, 10, or more than 10 peptide sequences) can be selected for further downstream processing according to their rank. In some embodiments, one or more peptides corresponding to the haplotypes identified in a sample may be selected using one or more predetermined selection criteria. The one or more predetermined selection criteria may each apply to the estimated haplotype prevalence, the estimated haplotype-transcript prevalence, a predicted likelihood of MHC binding, presentation, or immunogenicity, etc.

For example, in some embodiments the selected set of candidate tumor neoantigens (e.g., mutant peptide sequences) may then be inputted into one or more machine-learning models (e.g., one or more MHC binding and/or MHC presentation prediction models) to generate a prediction of a likelihood of presentation by an MHC for each of the set of candidate tumor neoantigens, or a prediction of an immunogenicity for each of the set of candidate tumor neoantigens. In certain embodiments, the prediction of the likelihood of presentation in an MHC for each of the set of candidate tumor neoantigens, or the prediction of immunogenicity for each of the set of candidate tumor neoantigens, may be then utilized to select and/or prioritize a subset of candidate neoantigens and/or to determine one or more individualized cancer immunotherapy treatments 224. For example, in one embodiment, the one or more individualized cancer immunotherapy treatments 224 may include genetically modified neoantigen-specific T-cells (e.g., primed T-cells) or a neoantigen vaccine (e.g., an RNA vaccine, or a DNA vaccine).

In some embodiments, the prediction of a likelihood of presentation and/or immunogenicity can be performed using NetMHC techniques (see, e.g., Nielsen et al. (2020), “Immunoinformatics: Predicting Peptide-MHC Binding”, Annu. Rev. Biomed. Data Sci. 3:191-215), which can predict binding of peptides to a number of different HLA alleles using artificial neural networks (ANNs) and weight matrices. Additionally or alternatively, predictions can be made based on the processes disclosed in, for example, in U.S. patent application Ser. No. 17/378,651 titled “ATTENTION-BASED NEURAL NETWORK TO PREDICT PEPTIDE BINDING, PRESENTATION, AND IMMUNOGENICITY” and U.S. Pat. Application No. 63/430,297 titled “PREDICTION OF PEPTIDE PRESENTATION BY MAJOR HISTOCOMPATIBILITY COMPLEX MOLECULES”, the contents of each of which are incorporated herein by reference. For example, the system can access a set of peptide sequences characterizing a set of peptides, each peptide sequence of the set of peptide sequences having been identified by processing a disease sample from a subject, access an immunoprotein complex (IPC) sequence identified for an immunoprotein complex (IPC) of the subject, and process a set of peptide representations that represents the set of peptide sequences using a first attention block in an initial attention subsystem of an attention-based machine-learning model and an immunoprotein complex (IPC) representation that represents the IPC sequence using a second attention block in the initial attention subsystem to generate an output including at least one of an interaction prediction, an interaction affinity prediction, or an immunogenicity prediction for a corresponding peptide-IPC combination. As another example, the system can predict an amino acid-immunoprotein complex (IPC) interaction by accessing a set of amino acid sequences, each of the amino acid sequences of the set having been identified from at least one protein, access an immunoprotein complex (IPC) sequence identified for an IPC of a subject. The system can then process, using one or more first processing blocks in a processing subsystem of a machine-learning model, a set of amino acid sequence representations to generate a set of transformed amino acid sequence representations based on a set of element-focused scores representing binding cores of the set of amino acid sequence representations, where each of the amino acid sequence representations was generated based on one of the amino acid sequences appended with a beginning-of-sequence (BOS) token. The system can process, using a second processing block in the processing subsystem, an IPC sequence representation to generate a transformed IPC sequence representation, where the IPC sequence representation was generated based on the identified IPC sequence appended with a BOS token, and where the set of amino acid sequence representations and the IPC sequence representation are processed in parallel. The system can generate composite representations by combining each of the transformed BOS token representations of the set of transformed amino acid sequence representations with the transformed BOS token representation of the transformed IPC sequence representation. The system can then determine one or more predicted amino acid-IPC interactions based on the composite representations.

FIG. 2B illustrates an exemplary computing platform 202 for performing phasing mutations in a tumor of a patient, in accordance with the presently disclosed embodiments. As depicted in FIG. 2B, the computing platform 202 may access data for a set of sequence reads 254 (e.g., in one or more sorted BAM files) generated by sequencing DNA and/or RNA extracted from one or more samples of the patient 210. In some embodiments, the set of sequence reads 254 may include tumor DNA sequence reads, tumor RNA sequence reads, normal DNA sequence reads, or a combination thereof. In certain embodiments, the set of sequence reads 254 can be generated utilizing one or more short read sequencing techniques and are thus of a relatively short length (e.g., as compared to long-read sequence reads).

In certain embodiments, as further depicted in FIG. 2B, the computing platform 202 may access or determine a plurality of genomic variant calls 256 (e.g., germline variant calls, somatic variant calls) related to the set of sequence reads 254 (e.g., tumor DNA sequence reads, tumor RNA sequence reads, and normal DNA sequence reads) to identify a plurality of germline and somatic variants 256. To determine the genomic variants 256, a variant calling process can be performed by the computing system 202 or by an upstream system in the pipeline. Non-limiting examples of suitable variant calling algorithms include HaplotypeCaller and Mutect2 (both from the Genome Analysis Toolkit, Broad Institute, Cambridge, MA) for calling germline variants and somatic mutations, respectively, from DNA sequence read data. Non-limiting examples of suitable variant calling algorithms for use with RNA sequence read data include Mpileup/Varscan from SAMtools (https://www.htslib.org/) and Haplotype Caller (e.g., after processing BAM files with the SplitNCigarReads (Genome Analysis Toolkit, Broad Institute, Cambridge, MA).

Mutation phasing as described herein may be performed independently on a subset of the mutations at a time. The disclosed methods determine which mutations need to be phased together for accurate neoantigen determination by defining phasing windows based on span lengths assigned to different types of mutations (as described below). This determination of phasing windows (and span lengths) in turn depends on a specified target neoantigen length (e.g., corresponding to amino acid sequences of about 21-27+ amino acid residues (to enable bound-peptide+flank modeling) or longer (i.e., nucleic acid sequences of about 63 to 81 bases, or longer) for peptides presented by MHC Class I molecules, or, for targeting MHC class II neoantigens, corresponding to amino acid sequences of about 15 to 34 amino acid residues or longer (i.e., nucleic acid sequences of about 45 to 102 bases, or longer)), and also depends on the type of upstream mutations identified. With respect to mutation type, accurate identification of neoantigens induced by a frameshift indel or stop-loss mutation can require phasing mutations that are located hundreds of bases apart from one another. For instance, mutations found in non-overlapping genes do not need to be phased together to infer possible neoantigen sequences, and so different genes may be separated into different phasing windows. Breaking the phasing problem into a series of small problems further increases the computational feasibility of the approach (while maintaining a sufficiently large phasing window to predict neoantigens), as a number of subprocesses (e.g. Bayesian inference) scale non-linearly with increasing number of mutations. Moreover, mutations that are farther apart, even in the same gene, may be difficult to phase with short-read sequencing data, and will likely not impact the sequence of a given relatively short (e.g., 21-27 amino acid long) neoantigen.

To minimize the number of variants (or mutations) phased at a time, the system can first break down the genome into phasing windows. To a first approximation, phasing windows correspond to gene sequences. In some cases, the system can include multiple genes with overlapping genomic coordinates in the same phasing window. Subsequently, the system can break down phasing windows further into phasing sub-windows based on the relative positions of variants identified in the DNA and/or RNA sequence read data. Phasing windows are defined by iteratively adding a span length specified for each variant (as described below) to a given “phasing window” definition until the phasing window stops overlapping with adjacent spans. Variants that are spaced far apart from one another in a given gene (e.g., that are associated with genomic coordinates that are separated by a distance that exceeds a specified distance threshold (e.g., a distance threshold ranging in value from about 63 bases to 81 bases)), and that thus reside in different phasing windows, often do not need to be phased together in order to infer the sequence of a short neoantigen (e.g., a peptide sequence of 21-27 amino acids in length). In some instances, for example, the system can determine variant coordinates in transcript space along with potential consequences of the variant. Spans of user-specified length are assigned to each somatic mutation coordinate (e.g., centered on the somatic mutation coordinate), and somatic mutations with overlapping spans can be assigned to the same phasing window or sub-window. The system can assign variants (somatic or germline) without overlapping spans to different phasing windows. For the purpose of neoantigen phasing, the system can assign germline variants a span of 1 nucleotide (i.e., to preclude them from unnecessarily expanding a phasing window) except in the following special cases. Frameshift variants, or variants that cause the loss or gain of a stop codon, whether germline or somatic in origin, can be assigned a lop-sided span where the downstream end of the span is set to the 3′-end of the transcript and the upstream end of the span is determined by the user-specified length of the span. This is due to the fact that the translation of variants downstream of these special case variants can be dependent on whether or not they are in phase with the frameshift/stop loss/gain variants.

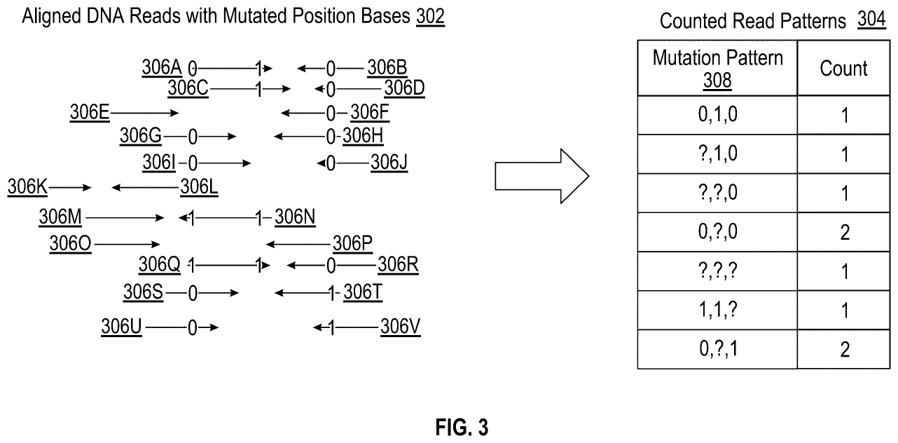

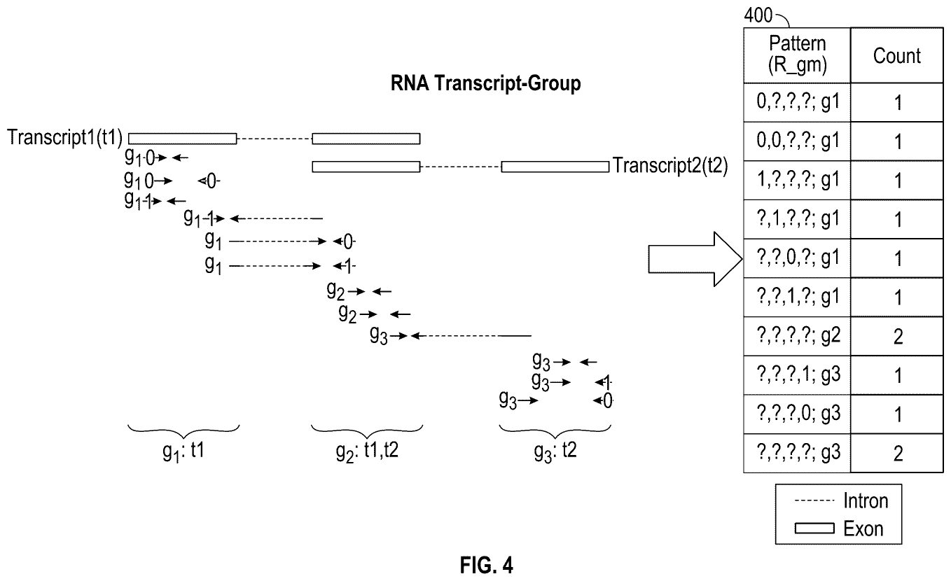

As further illustrated by FIG. 2B, the computing platform 202 may include a sequence read counter (or read pattern counter) 258. The sequence read counter 258 may identify unique mutation patterns observed in the sequence reads 254 and quantify or count each unique mutation pattern observed in the sequence reads 254. A mutation pattern indicates a specific combination of variant alleles (which may include the ‘unobserved’ allele). One exemplary unique mutation pattern may be “1, 0, 1”, which indicates an allele value “1” at a first base position, an allele value “0” at a second base position, and an allele value “1” at a third base position. Another exemplary unique mutation pattern may be “1, 1, 1”, which indicates an allele value “1” at the first base position, an allele value “1” at the second base position, and an allele value “1” at the third base position. A “?” character is used to denote when a variant's state is unobserved in a given read. For each identified unique mutation pattern, the system can further count how many times the unique mutation pattern has been observed in the sequence reads. For example, the system can count that the unique mutation pattern “1, 1, 1” has appeared in two sequence reads, and thus determines that the quantity of the mutation pattern “1, 1, 1” to be 2. Accordingly, the system can enumerate one or more unique mutation patterns (by generating all possible combinations of the mutations identified in the sequence read data) and, for each unique mutation pattern, determine an associated mutation pattern quantity (by counting the number of sequence reads (DNA and/or RNA sequence reads) that exhibit a given unique mutation pattern). In a similar manner, the system can enumerate combinations of a set of unique mutation patterns and a set of transcript groups observed in RNA sequence reads (as described above), and determine an associated mutation pattern-transcript group quantity (by counting the number of RNA sequence reads assigned to the specified transcript group that exhibit the specified unique mutation pattern). Details of the sequence read counter are provided below with reference to FIGS. 3-4.

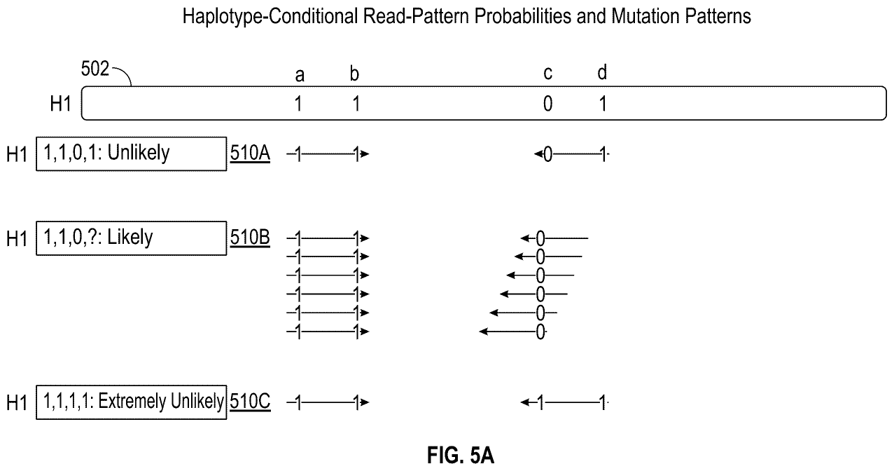

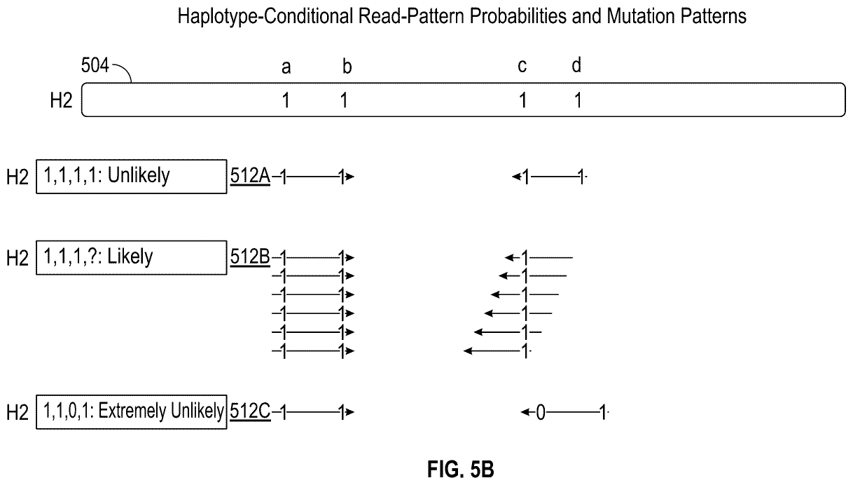

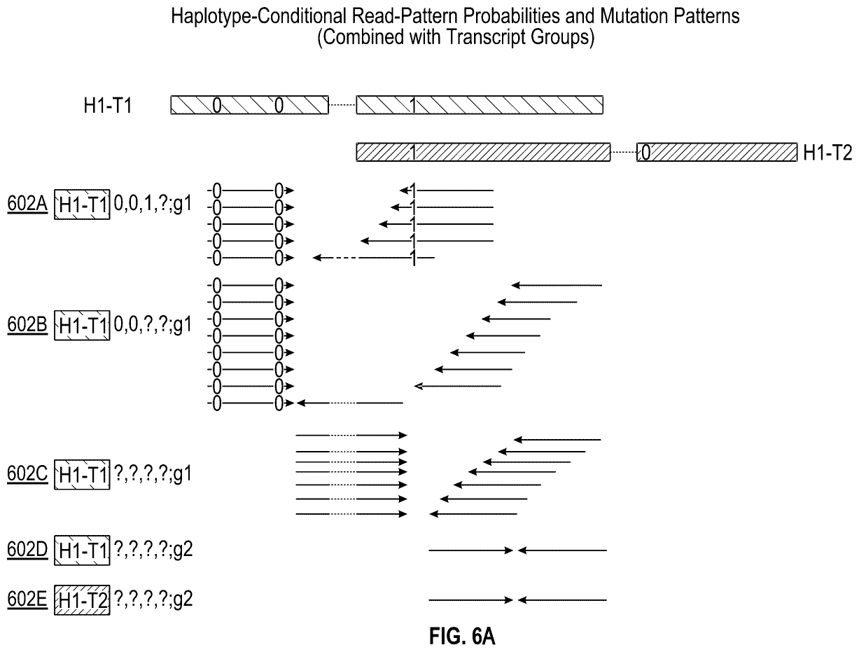

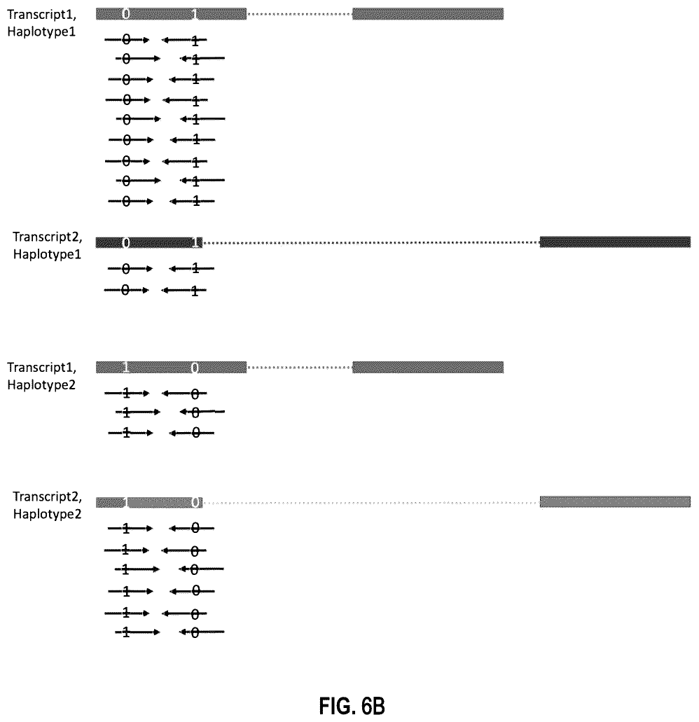

As further illustrated by FIG. 2B, the computing platform 202 may include an enumeration and pattern probability estimator 260. The enumeration and pattern probability estimator 260 determines, for each of the unique mutation patterns, a probability that a hypothetical DNA sequence read and/or RNA sequence read from each one of a set of haplotypes will exhibit the unique mutation pattern. For example, with respect to haplotype A, the system can determine a first probability indicative of how likely a hypothetical DNA sequence read and/or RNA sequence read from the haplotype A will exhibit the unique mutation pattern “1, 0, 1.” Further, the system can determine a second probability indicative of how likely a hypothetical DNA sequence read and/or RNA sequence read from the haplotype A will exhibit the unique mutation pattern “1, 1, 1.” Accordingly, a plurality of probabilities can be determined for a given haplotype corresponding to a plurality of unique mutation patterns (where the given haplotype is taken from a plurality of candidate haplotypes generated by taking all possible combinations of mutations within a gene or gene region and their associated alleles). In a similar manner, the system can determine a probability, for each one of a set of combinations of the haplotypes and a set of transcript groups associated with that haplotype, that a hypothetical RNA sequence from a specific haplotype and transcript pair will exhibit the unique mutation pattern and transcript group label (where the transcript group label indicates the transcript group to which the RNA sequence read is assigned). Details of the enumeration and pattern probability estimator are provided below with reference to FIGS. 5A-6.

As further illustrated by FIG. 2B, the computing platform 202 may include a statistical model 262 such as a hierarchical Bayesian model. Based on the mutation pattern/mutation pattern-transcript-group quantities (obtained by the sequence read counter 258) and the haplotype/haplotype-transcript conditional mutation pattern probabilities (obtained by the enumeration and pattern probability estimator 260), the statistical model 262 may be used to estimate output 264, which includes one or more of a set of probabilities that each of the haplotypes exists (also referred to as haplotype-existence probabilities), the set of haplotype prevalences, and/or the set of haplotype-transcript prevalences. Details of exemplary models are provided below with reference to FIG. 7.

As further depicted in FIG. 2, the system 200 can identify a set of peptide sequences using translations 266 of one or more haplotype transcripts based on the set of haplotype-existence probabilities. In some embodiments, the system identifies a set of mutant peptide sequences based on the set of peptide sequences. In some embodiments, the system can determine which haplotypes are present based on the haplotype-existence probabilities (e.g., by determining if the probability is non-zero or exceeds a predefined threshold). Those haplotypes that are present can be translated into peptide sequences. In other words, the system can limit the downstream analysis on those haplotypes that are known to be present based on the sequence read data so as to avoid targeting a haplotype that is not present since it would not be effective as a treatment. The prevalences are used to select a subset of the peptide sequences to target in a vaccine. For example, if the prevalence estimate for a given haplotype is greater than a threshold value, the corresponding peptide sequence can be selected for further downstream processing. On the other hand, if the prevalence estimate for a given haplotype is lower than the threshold value (e.g., indicative of very low prevalence), the system can exclude the haplotype for downstream process so as to avoid targeting in a vaccine a haplotype with a low prevalence. Alternatively, the peptide sequences can be ranked at least in part according to their prevalences, and some subset of those peptide sequences can be selected for further downstream processing according to their rank.

In certain embodiments, one or more machine-learning models (e.g., one or more MHC binding and/or MHC presentation prediction models) may generate a prediction of a likelihood of presentation in an MHC for one or more of the set of mutant peptide sequences, or a prediction of an immunogenicity for one of more of the set of mutant peptide sequences. The prediction of the likelihood of presentation in a MHC for one or more of the set mutant peptide sequences, or the prediction of an immunogenicity for one or more of the set of mutant peptide sequences may be then utilized to identify one or more candidate neoantigens and determine one or more individualized cancer immunotherapy treatments, such as one or more genetically modified neoantigen-specific T-cells (e.g., primed T-cells) or a neoantigen vaccine (e.g., an RNA vaccine, or a DNA vaccine).

### III. Sequence Read Counter

### III.A Sequence Read Counter—DNA Reads

FIG. 3 illustrates exemplary processing of the exemplary sequence read counter 258 for enumerating and quantifying unique mutation patterns observed in sequence read data (e.g., for DNA sequence reads), in accordance with the presently disclosed embodiments. In these examples, the weighted counting process described below is ignored for simplicity. The counter 258 accesses data for a plurality of aligned (DNA and/or RNA) sequence reads 302. The aligned sequence reads 302 may be part of the sequence read data 254 and variant calls 256 in FIG. 2A.

In the depicted example, the sequencing data comprises data for sequence read pairs. Sequencing can involve single-end (SE) or paired-end (PE) reads. Paired-end sequencing entails sequencing both ends of a DNA fragment. The forward and reverse reads can then be aligned. As described herein, a sequence read may comprise a pair of two reads or a read pair, which includes a forward read and a reverse read, as shown in FIG. 3.

With reference to FIG. 3, the sequence read counter analyzes the sequence reads 302 to identify unique mutation patterns 308. As an example, the plurality of aligned (DNA and/or RNA) sequence reads 302 may indicate a plurality of mutation patterns 306A-306V. For example, as depicted by FIG. 3:
aligned sequence read pair 306A and 306B indicates a mutation pattern “0, 1, 0”;
aligned sequence read pair 306C and 306D indicates a mutation pattern “?, 1, 0”;
aligned sequence read pair 306E and 306F indicates a mutation pattern “?; ?; 0”;
aligned sequence read pair 306G and 306H indicates a mutation pattern “0, ?, 0”;
aligned sequence read pair 306I and 306J indicates a mutation pattern “0, ?, 0”;
aligned sequence read pair 306K and 306L indicates a mutation pattern “?, ?, ?”;
aligned sequence read pair 306M and 306N indicates a mutation pattern “?, 1, 1”;
aligned sequence read pair 306O and 306P indicates a mutation pattern “?, ?, ?”;
aligned sequence read pair 306Q and 306R indicates a mutation pattern “1, 1, 0”;
aligned sequence read pair 306S and 306T indicates a mutation pattern “0, ?, 1”; and
aligned sequence reads 306U and 306V indicate a mutation pattern “0, ?, 1”.

In the depicted example in FIG. 3, each time the sequence read counter identifies a unique mutation pattern (e.g., a pattern in column 308) in the aligned (DNA or RNA) sequence reads 302, the sequence read counter can update the table 304 (or database, or other preferred memory structure) to include the identified mutation pattern and the number of times the mutation pattern is counted for the rest of the reads for the associated sample(s) of the subject. For example:
mutation pattern “0, 1, 0” is observed once in aligned read pair 306A-B and thus counted as 1;
mutation pattern “?, 1, 0” is observed once in aligned read pair 306C-306D and thus counted as 1;
mutation pattern “?, ?, 0” is observed once in aligned read pair 306E-F and thus counted as 1;
mutation pattern “0, ?, 0” is observed twice in aligned read pair 306G-H and then aligned reads 306I-J and thus counted as 2;
mutation pattern “?, ?, ?” is observed once in aligned read pair 306K-L and thus counted as 1;
mutation pattern “1, 1, ?” is observed once in aligned read pair 306Q-R and thus counted as 1; and
mutation pattern “0, ?, 1” is observed in aligned read pair 306S-T and then aligned read pair 306U-V and thus counted as 2.
   For tumor DNA sequence reads, the number of sequence reads exhibiting each unique mutation pattern are counted. For tumor RNA sequence reads, the number of sequence reads that exhibit each combination of unique mutation pattern and transcript group are counted. DNA and RNA counting operations are performed separately when both DNA sequence read and RNA sequence read data are utilized. In some embodiments, the number of normal-tissue-sample DNA sequence reads that exhibit each unique mutation pattern is also counted, and the phasing of normal haplotypes is estimated using a DNA-only model. This data provides information for the normal proteome, which can then be used to ensure that candidate tumor-associated neoantigens are truly tumor-specific targets. Moreover, haplotypes identified in normal data can be input to the tumor-phasing model as a necessary part of the tumor phasing solution (to account for contamination).

### III.B Sequence Read Counter—RNA Reads

The sequence read counter can also be used to count how many sequence reads are in a particular RNA transcript group, in accordance with some embodiments. FIG. 4 illustrates exemplary processing of the exemplary sequence read counter 258 for enumerating and quantifying unique mutation patterns observed in RNA sequence reads, in accordance with the presently disclosed embodiments.

With reference to FIG. 4, the sequence read counter analyzes the sequence reads to identify unique combinations of mutation patterns and transcript groups. In the depicted example, the plurality of reads give rise to the following unique combinations of mutation pattern and transcript group:
0,?,?.?; g1
0,0,?,?; g1
1,?,?,?; g1
?,1,?,?; g1
?,?,0,?; g1
?,?,1,?; g1
?,?,?,?; g2
?,?,?,1; g3
?,?,?,0; g3
?,?,?,?; g3

In the depicted example in FIG. 4, each time the sequence read counter identifies a unique combination of mutation pattern and transcript group, the sequence read counter can update the table 400 (or database, or other preferred memory structure) to include the identified combination and track the number of occurrence for that identified combination. For example:
The combination of mutation pattern “0, ?, ?, ?” and g1 is observed once and thus counted as 1;
The combination of mutation pattern “0, 0, ?, ?” and g1 is observed once and thus counted as 1;
The combination of mutation pattern “1, ?, ?, ?” and g1 is observed once and thus counted as 1;
The combination of mutation pattern “?, 1, ?, ?” and g1 is observed once and thus counted as 1;
The combination of mutation pattern “?, ?, 0, ?” and g1 is observed once and thus counted as 1;
The combination of mutation pattern “?, ?, 1, ?” and g1 is observed once and thus counted as 1;
The combination of mutation pattern “?, ?, ?, ?” and g2 is observed twice and thus counted as 2;
The combination of mutation pattern “?, ?, ?, 1” and g3 is observed once and thus counted as 1;
The combination of mutation pattern “?, ?, ?, 0” and g3 is observed once and thus counted as 1;
The combination of mutation pattern “?, ?, ?, ?” and g3 is observed twice and thus counted as 2.

### IV. Enumeration and Pattern Probability Estimator

### IV.A. DNA Reads

The enumeration and pattern estimator, for each haplotype, h, enumerates all possible sequence reads that could be generated for the haplotype (e.g., by shifting a sequence read of a specified length along the haplotype sequence one base at a time) and performs a weighted summation of the number of those sequence reads that would exhibit a given unique mutation pattern, m (where the weights account for sequencing error and insert length). The enumerated and weighted-counted sets of sequence reads are then used to determine a probability, Wₘₕ, corresponding to the un-normalized numerator of the probability that a hypothetical DNA sequence read from that haplotype will exhibit the unique mutation pattern (this quantity can be normalized by dividing the weighted number of sequence reads expected to exhibit the unique mutation pattern by the total weighted number of possible sequence reads that could be generated for the haplotype irrespective of mutation pattern; the normalizing constant cancels out during model computation, so is not necessary). Turning back to FIG. 2B, the enumeration and pattern probability estimator 260 determines, for each of the unique mutation patterns, the unnormalized probability that a hypothetical DNA sequence read from each one of the set of haplotypes will exhibit the unique mutation pattern. For example, with respect to haplotype A, the system can determine a first unnormalized probability indicative of how likely a hypothetical DNA sequence read from the haplotype A will exhibit the unique mutation pattern “1, 0, 1.” Further, the system can determine a second unnormalized probability indicative of how likely a hypothetical DNA sequence read from the haplotype A will exhibit the unique mutation pattern “1, 1, 1.” Accordingly, a plurality of unnormalized probabilities can be determined for a given haplotype corresponding to a plurality of unique mutation patterns.

FIGS. 5A-B illustrate exemplary processing by the enumeration and pattern probability estimator 260 for determining a probability that a hypothetical DNA sequence read from a given haplotype will exhibit a given unique mutation pattern, in accordance with the presently disclosed embodiments. The estimator accesses one or more theoretical example haplotypes 502 (“H1”) in FIG. 5A and 504 (“H2”) in FIG. 5B (where the one or more theoretical example haplotypes are specific combinations of the set of mutations and their associated alleles identified in the plurality of sequence reads). The example haplotype 502 (H1) may include alleles “1” at a first base position a, “1” at a second base position b, “0” at a third base position c, and “1” at a fourth base position c, while the example haplotype 504 (H2) may include alleles “1” at the first base position a, “1” at the second base position b, “1” at the third base position c, and “1” at the fourth base position d. The estimator determines a probability indicative of the likelihood that a hypothetical DNA sequence read from a given haplotype will exhibit a given unique mutation pattern, as described below.

As shown, the estimator 260 can determine a probability 510A indicative of the likelihood that a hypothetical DNA sequence read from haplotype 502 (H1) will exhibit a unique mutation pattern “1, 1, 0, 1” in the sequence reads. The probability 510A may be a relatively low probability indicative of an unlikely event, as a sequence read pair including alleles “1” at base position a, “1” at base position b, “0” at base position c, and “1” at base position d would have to be observed where the sequence read pair has the exact matched order and encompasses a read pair of sufficient positioning and/or length with respect to the “1” “1” “0” “1” allele pattern present in the example haplotype 502 (H1). The example of FIG. 5A illustrates this as a low probability event by showing the only (in this example) possible such ordering and position and/or length of a hypothetical sequence read from capturing such an allele pattern.

As another example, the estimator 260 can determine a probability 510B indicative of the likelihood that a hypothetical DNA sequence read pair from haplotype 502 (H1) will exhibit a unique mutation pattern “1, 1, 0, ?”. The probability 510B may be higher than the probability 510A, indicative of a more likely event as haplotype 502 can, for many more positions, yield read pairs exhibiting “1, 1, 0, ?”. The “?” provides the right read of the pair a greater degree of freedom in its placement, as it now no longer covers the last mutation position. The example of FIG. 5A illustrates this as a relatively higher probability event by showing there are six hypothetical sequence read pairs having such ordering and position and/or length for capturing such an allele pattern.

As another example, the estimator can determine a probability 510C indicative of the likelihood that a hypothetical DNA sequence read pair from haplotype 502 (H1) will exhibit a unique mutation pattern “1, 1, 1, 1” in the sequence reads. The probability 510C may be lower than the probability 510A, indicative of an extremely unlikely event, as a sequence read pair including alleles “1” at base position a, “1” at base position b, “1” at base position c, and “1” at base position d could not possibly match to the “1” “1” “0” “1” allele pattern present in the example haplotype 502 (H1) with respect to both order and position, without some form of sequencing error at the third allele, which is possible but rare. The example of FIG. 5A illustrates this as a low probability event by showing the only (in this example) possible such ordering and position and/or length of a hypothetical sequence read from capturing such an allele pattern.

Similarly, as further illustrated in FIG. 5B, for the example haplotype 504 (H2), various probabilities can be determined by the estimator. For example, the estimator can determine a probability 512A indicative of the likelihood that a hypothetical DNA sequence read pair from haplotype 504 (H2) will exhibit a unique mutation pattern “1, 1, 1, 1” in the sequence reads. The probability 512A may be a relatively low probability indicative of an unlikely event, as a sequence read pair including alleles “1” at base position a, “1” at base position b, “1” at base position c, and “1” at base position d would have to be observed at the exact matched order and position with respect to the “1” “1” “1” “1” allele pattern present in the example haplotype 504 (H2). The example of FIG. 5B illustrates this as a low probability event by showing the only (in this example) possible such ordering and position and/or length of a hypothetical sequence read for capturing such an allele pattern.

As another example, the estimator can determine a probability 512B indicative of the likelihood that a hypothetical DNA sequence read pair from haplotype 504 (H2) will exhibit a unique mutation pattern “1, 1, 1, ?” in the sequence reads. The probability 512B may be higher than the probability 512A, indicative of a more likely event, as a sequence read pair including alleles “1” at base position a, “1” at base position b, “1” at base position c, and “?” at base position d is more likely to be observed at the exact matched order and position with respect to at least the first three alleles of the “1” “1” “1” “1” allele pattern present in the example haplotype 504 (e.g., H2). The example of FIG. 5B illustrates this as a relatively higher probability event by showing there are six hypothetical sequence reads having such ordering and position and/or length for capturing such an allele pattern.

As another example, the estimator can determine a probability 512C indicative of the likelihood that a hypothetical DNA sequence read from haplotype 504 (H2) will exhibit a unique mutation pattern “1, 1, 0, 1” in the sequence reads. The probability 512C may be lower than the probability 512A, indicative of an extremely unlikely event, as a sequence read pair including alleles “1” at base position a, “1” at base position b, “0” at base position c, and “1” at base position d could not possibly match to the “1” “1” “1” “1” allele pattern present in the example haplotype 504 (H2) with respect to both order and position without some form of sequencing error at the third allele, which is possible but rare. The example of FIG. 5B illustrates this as a low probability event by showing the only (in this example) possible such ordering and position and/or length of a hypothetical sequence read for capturing such an allele pattern.

### IV.B. RNA Reads

In certain embodiments, the enumeration and pattern probability estimator 210 may be further utilized to determine, for each of the unique mutation pattern m, an unnormalized probability, W_{gmht}, that a hypothetical RNA sequence from each combination of a set of combinations of haplotypes and transcripts will exhibit the unique mutation pattern m and transcript group g, when arising from a molecules of haplotype h, and the transcript t. The probability, W_{gmht}, can be determined, for example, by enumerating all possible RNA sequence reads that could be generated for haplotype h, and transcript t, by shifting an RNA sequence read of a specified length along the haplotype sequence one base at a time, performing a weighted summation (the weights based again on insert length and the amount of sequencing error) of the number of these RNA sequence reads that would be consistent with transcript t, and that would exhibit transit group, g, and the specific mutation pattern m (this could again be normalized by dividing by the weighted number of RNA reads that could arise from haplotype h and transcript t, irrespective of mutation pattern or transcript group).

In the example depicted in FIG. 6A, a haplotype H1 is associated with two transcripts: T1 and T2. The two transcript groups give rise to three transcript groups, g1, g2, and g3, as discussed above with reference to FIG. 1E. Accordingly, for the combination of H1 and T1, the system can determine a probability that a hypothetical RNA sequence from that H1 will exhibit a combination of a given unique mutation pattern and one of the transcript groups. In the example depicted in FIG. 6A, the system can determine:

for the combination of H1 and T1
a probability 602A that a hypothetical RNA sequence from that H1 and T1 will exhibit a combination of mutation pattern “0, 0, 1, ?” and transcript group g1;
a probability 602B that a hypothetical RNA sequence from that H1 and T1 will exhibit a combination of mutation pattern “0, 0, ?, ?” and transcript group g1;
a probability 602C that a hypothetical RNA sequence from that H1 and T1 will exhibit a combination of mutation pattern “?, ?, ?, ?” and transcript group g1;
a probability 602D that a hypothetical RNA sequence from that H1 and T1 will exhibit a combination of mutation pattern “?, ?, ?, ?” and transcript group g2; and
any other probabilities (not depicted) that a hypothetical RNA sequence from that H1 and T1 will exhibit a combination of a particular mutation pattern and one of transcript groups g1, g2, and g3.

For the combination of H1 and T2
a probability 602E that a hypothetical RNA sequence from that H1 and T2 will exhibit a combination of mutation pattern “?, ?, ?, ?” and transcript group g2; and
any other probabilities (not depicted) that a hypothetical RNA sequence from that H1 and T2 will exhibit a combination of a particular mutation pattern and one of transcript groups g1, g2, and g3.

As shown in FIG. 6A, each of probabilities 602A-E can be determined by determining how many hypothetical sequence reads may have such ordering and position and/or length for capturing such an allele pattern. The example of FIG. 6A illustrates that probability 602B as a relatively higher probability event by showing there are eight hypothetical sequence reads having such ordering and position and/or length for capturing such an allele pattern. In contrast, probability 602E is a relatively lower probability event because there is only one hypothetical sequence read having such ordering and position and/or length for capturing such an allele pattern.

In other words, for every haplotype-transcript (ht), for every combination (gm) of a transcript group and a mutation pattern, the system can count the number of possible read pairs that can result in such combination gm. In some embodiments, the system weights the counted read pairs by insert length probability and/or the probability of a sequencing error, as described below.

FIG. 6B provides a schematic illustration of the utility of determining haplotype-transcript prevalence when selecting tumor-associated peptides or proteins for the development of, e.g., personalized anti-cancer therapies. Each haplotype-transcript combination encodes a different protein. The only protein with high prevalence (as indicated in the figure by the relative number of aligned RNA sequence reads) is the one encoded by transcript1-haplotype1 (T1-H1). T2-H1 and T1-H2 have low expression. T2-H2 has medium expression. If selection of proteins to target was based solely on protein expression, T1-H1 would be the best target. Also note that even though T1 total expression—independent of haplotype—is high, T1-H2 is not a good target. This illustrates why determination of transcript-specific expression can be inadequate for selection of proteins (e.g., tumor antigens) for downstream treatment development, and why the ability to determine transcript-haplotype specific expression (based on determination of transcript-haplotype prevalence) is a more useful selection criterion.

### V. Statistical Model for Performing Phasing Analysis

### V.A. Expressions for Performing Phasing Analysis

For DNA sequence reads, the probability of observing mutation pattern m depends on (1) the prevalence of each haplotype (h), and (2) the haplotype-conditional probability of observing the mutation pattern m in a given read. This relationship can be represented as:

$$\begin{matrix}\begin{matrix}{p}_{m} = \frac{{\sum }_{hlja} I ( h , l , j , a ) \times {δ}_{a m}^{d} \times {ϕ}_{h}^{d} \times p ( {l}^{d} )}{{\sum }_{{hljam}^{′}} I ( h , l , j , a ) \times {δ}_{a {m}^{′}} \times {ϕ}_{h}^{d} \times p ( {l}^{d} )} \\ = \frac{{\sum }_{h} {W}_{m h} \times {ϕ}_{h}^{d}}{{\sum }_{{hm}^{′}} {W}_{{m}^{′} h} \times {ϕ}_{h}^{d}}\end{matrix} \\ \end{matrix}$$

where pₘ is the probability of observing a DNA sequence read that exhibits unique mutation pattern m.

In the expression above, I(h, l, j, a) is, as described earlier, an indicator denoting whether a DNA read from haplotype h starting at position j, with insert length l, and actual allele a-will exhibit mutation pattern m.

$${ϕ}_{h}^{d}$$

represents the unknown haplotype prevalence to be solved by the statistical model 262, as discussed below. Wₘₕ (designated as

$${W}_{m h}^{d}$$

in FIG. 7 to indicate a value determined for DNA by Enumeration and Pattern Probability Estimator 260 in FIG. 2B) is proportional (without the normalization constant) to the probability that a read from a DNA molecule of haplotype h would exhibit mutation pattern m. Wₘₕ is computed independently of any actual sequence reads; it depends on mutation positions, a DNA sequencing error model, and a read insert length probability model.

As descried above,

$${δ}_{a m}^{d}$$

is the probability that a DNA read having actual mutation pattern a will be sequenced as though it were mutation pattern m. p^{d}(l) is the probability that a DNA read pair has insert length l. As described earlier, this parameter is determined by fitting a frequentist negative binomial model to DNA reads from across the entire genome.

In the denominator of the expression above, Wₘₕ is summed over all mutation patterns m′, whereas in the numerator, Wₘₕ is summed over haplotype h to count the number of ways that a DNA sequence read can be generated for a specific mutation pattern m.

For RNA sequence reads, the probability of observing the combination of the mutation pattern m and transcript group g in a given read depends on (1) the prevalence of each haplotype-transcript combination, (2) the haplotype-transcript-conditional probability of observing the combination of mutation pattern m and transcript group g, and (3) distance between mutations in transcript space. This relationship can be expressed as:

$$\begin{matrix}\begin{matrix}{p}_{gm} = \frac{{\sum }_{htlja} I ( h , t , l , j , g , a ) \times {δ}_{a m}^{r} \times {ϕ}_{ht}^{r} \times p ( {l}^{r} )}{{\sum }_{{htljag}^{′} {m}^{′}} I ( h , t , l , j , {g}^{′} , a ) \times {δ}_{{am}^{′}}^{r} \times {ϕ}_{ht}^{r} \times p ( {l}^{r} )} \\ = \frac{{\sum }_{ht} {W}_{gmht} \times {ϕ}_{ht}^{r}}{{\sum }_{{htg}^{′} {m}^{′}} {W}_{{g}^{′} {m}^{′} ht} \times {ϕ}_{ht}^{r}}\end{matrix} \\ \end{matrix}$$

where p_{gm} is the probability of observing an RNA sequence read that exhibits unique mutation pattern m in transcript group g.

In this expression, I(h, t, l, j, g, a) indicates whether it is possible for haplotype h and transcript t to generate a read of insert length l, with read1 (i.e., the leftmost read of a read pair) and start j (the genomic coordinate of the start of the left-most read of the read pair), in transcript group g, and with an actual mutation pattern a (as opposed to observed mutation pattern m).

$${δ}_{a m}^{r}$$

indicates the probability of miscalling a base or set of bases in an RNA read having actual mutation pattern a with the observed mutation pattern m (due to sequencing error). In other words, the probability that the hypothetical RNA sequence read from that haplotype will exhibit the unique mutation pattern in the combination of the haplotype and the transcript group can be based on a probability of miscalling tumor RNA sequence reads with the unique mutation pattern. In some embodiments, δₐₘ can be estimated this by examining all reads simultaneously to determine the rate at which single-base mismatches of type allele a to allele m are observed at positions within the aligned read which have not already been called as mutations. In some embodiments, equal error probability can be assumed irrespective of the class of error. A similar approach may be taken to model sequencing error for DNA.

p^{r}(l) indicates the probability of insert length l (e.g., the distance between the start of read1 and end of read2 for a paired-end sequence read). In other words, the probability that the hypothetical RNA sequence read from that haplotype will exhibit the unique mutation pattern in the combination of the haplotype and the transcript group can further be based on a probability of an insert length for the RNA sequence read with the unique mutation pattern. In some embodiments, for RNA sequence read insert length modeling, very long exons are identified and the observed insert lengths from sequence reads from those very long exons are fit to a negative binomial model. In some embodiments, for DNA sequence read insert length modeling, all observed insert lengths are fit to a negative binomial model. P(l) is then computed using the fit negative binomial.

In the expression above, φₕₜ^{r} represents the unknown prevalence of RNA molecules of haplotype h, transcript t, to be solved by the statistical model 262, as discussed below. W_{gmht} (designated as

$${W}_{g m h t}^{r}$$

in FIG. 7 to indicate a value determined for RNA by Enumeration and Pattern Probability Estimator 260 in FIG. 2B) is the unnormalized probability, conditional on haplotype h and transcript t, of observing the combination of mutation pattern m and transcript group g in a given sequence read determined from the RNA sequence read data, as described above. W_{gmht} is computed independently of any actual sequence reads; it depends on mutation positions, on exons defined by the GTF file, on an RNA sequencing error model, and on a read insert length probability model.

W_{g′m′ht} is the haplotype-transcript unnormalized conditional probability of observing the combination of mutation pattern m′ and group g′ to be summed over all possible combinations of mutation pattern m′ and group g′.

Note that determining pₘ, the probability of observing a DNA sequence read that exhibits unique mutation pattern m, can be considered a special case of determining p_{gm} in which there is effectively only 1 transcript and only 1 transcript group, so there is no need to index over g or t. Sequencing error and insert length distributions for RNA reads versus DNA reads may differ.

### V.B. Graph Representation of a Statistical Model for Performing Mutation Phasing

FIG. 7 illustrates an exemplary statistical model 262 for estimating a set of probabilities that each of a number of haplotypes exists and gave rise to a set of sequence reads (also referred to as haplotype-existence probabilities), as well as a set of haplotype prevalences and haplotype-transcript prevalences, in accordance with the presently disclosed embodiments. Specifically, in accordance with the presently disclosed embodiments, the statistical model 262 is used to estimate a set of probabilities (also referred to as haplotype-existence probabilities) that each of a number of haplotypes exists and gave rise to observed sequence reads (e.g., tumor DNA sequence reads, tumor RNA sequence reads, and normal DNA sequence reads) as well as a set of haplotype prevalences and haplotype-transcript prevalences based on the outputs of the read pattern counter 258 (i.e., the counts of different mutation patterns, the counts of different combinations of mutation patterns and transcript groups) and the outputs of the enumeration and pattern probability estimator 260 (i.e., the various conditional probabilities described above). In other words, the system uses the read pattern counter 258 to count occurrences of mutation patterns and/or mutation-pattern-transcript-group combinations, uses the enumeration and pattern probability estimator 260 to determine conditional probabilities of these mutation patterns or mutation-pattern-transcript-group combinations under different haplotype scenarios, and uses the statistical model 262 to estimate the most likely scenarios, as discussed below.

In some embodiments, statistical model (or probabilistic graphical model) 262 may include a haplotype generation model, a DNA haplotype prevalence model (e.g., a DNA Dirichlet-Multinomial model 704, as illustrated in the non-limiting example shown in FIG. 7), and/or a haplotype-transcript group model (e.g., an RNA Dirichlet-Multinomial model 706 as illustrated in the non-limiting example shown in FIG. 7). The haplotype generation model uses the unique mutation pattern sequence read counts and mutation pattern probabilities determined from sequence read count data as part of the upstream analysis described above to determine haplotype existence probabilities for a given sample. The DNA haplotype prevalence model uses the set of haplotype existence probabilities determined by the haplotype generation model and the conditional probability that a hypothetical DNA sequence read from a given haplotype will exhibit a specified unique mutation pattern to estimate haplotype prevalences in the sample. The haplotype-transcript group model uses the set of haplotype existence probabilities determined by the haplotype generation model and the conditional probability that a hypothetical RNA sequence read from a given haplotype and transcript group will exhibit a specified unique mutation pattern to estimate haplotype-transcript group prevalences in the sample.

In some embodiments, for example, the statistical model 262 may include a haplotype generation model 702, a DNA Dirichlet-Multinomial model 704, and/or an RNA Dirichlet-Multinomial model 706. In certain embodiments, the haplotype generation model 702, the DNA Dirichlet-Multinomial model 704, and the RNA Dirichlet-Multinomial model 706 may each include one or more directed acyclic graphs (DAGs), which may further include respective nodes 708, 710, 712, 714, 716, 718, 720, 722, 724, and 726 interconnected by one or more directed links and being associated with one or more discrete values estimated from a range of probability distributions associated with each of the respective nodes 708, 710, 712, 714, 716, 718, 720, 722, 724, and 726.

The haplotype generation model 702 may include a normal haplotypes node 708 (e.g., Nₕ), a tumor-specific haplotypes node 710 (e.g., Xₕ), and a tumor sample haplotypes node 712 (e.g., Hₕ). Hₕ of the tumor sample haplotypes node 712 is an indicator variable denoting whether or not haplotype h is present in the sample. The system can estimate a set of haplotype-existence probabilities by estimating Hₕ. As discussed below, by fitting the model, the system can estimate a posterior distribution of Hₕ (i.e., a distribution of whether haplotype h is in the sample or not in the sample).

The normal haplotypes node 708 (e.g., Nₕ) may include an estimated or approximated binary value (e.g., a posterior distribution estimated or approximated based on a range of probability distributions associated with the normal haplotypes node 708), in which Nₕ=“1” if a given normal haplotype is present in one or more samples of the subject and Nₕ=“0” if a given normal haplotype is not present. Nₕ may be drawn from one or more samples of the subject. In some embodiments, Nₕ may be estimated using other phasing tools. In some embodiments, somatic mutation-free haplotypes that are found in normal tissue are also likely to be found in the tumor tissue due to unavoidable contamination of the tumor sample by normal cells. In some embodiments, this can be accounted for by giving a very high prior to such haplotypes. In some instances, germline variant phasing is likely to be conserved in tumor haplotypes that additionally include some somatic mutation. When this is true, one can give higher prior probability to tumor haplotypes that preserve germline variant structure.

The tumor-specific haplotypes node 710 (e.g., Xₕ) may include an estimated or approximated binary value (e.g., a posterior distribution estimated or approximated based on a range of probability distributions associated with the tumor-specific haplotypes node 710), in which Xₕ=“1” if a given tumor-specific haplotype is present in the one or more samples of the subject and Xₕ=“0” if a given tumor-specific haplotype is not present in the one or more samples of the subject.

In certain embodiments, the normal haplotypes node 708 (e.g., Nₕ) and the tumor-specific haplotypes node 710 (e.g., Xₕ) may be connected by way of respective directed links to the tumor sample haplotypes node 712 (e.g., Hₕ). Hₕ is a mixture of normal haplotypes Nₕ and tumor haplotypes Xₕ. Thus, in one embodiment, the tumor sample haplotypes node 712 (e.g., Hₕ) may include an estimated or approximated binary value (e.g., estimated or approximated based on the union of the values Nₕ and Xₕ), in which Hₕ=“1” if either Nₕ=“1” or Xₕ=“1” indicating that at least one of a normal haplotype or a tumor-specific haplotype is present in the bulk tumor sample. In certain embodiments, one or more hyperpriors may be represented by a value “π” (connected to Xₕ), which may be utilized to incorporate any prior knowledge of the bulk tumor sample into the haplotype generation model 702. In some embodiments, for example, a weak hyperprior on Bernoulli variables may be used. As discussed below, the system can use the hyperprior to sample tumor haplotypes (and optionally normal haplotypes). Normal haplotype phasing can be used to adjust the prior/hyperprior on the existence of tumor haplotypes. In some embodiments, the normal haplotypes are determined based on the use of a preprocessing tool (e.g., HapCUT2, WhatsHap). As another alternative, the system can mix and match possibilities of where Nₕ and Xₕ come from; for example, normal haplotypes could be specified as known quantities measured by some other method; e.g., long-reads, microarray data, mother-father-child trio information, dilution pool sequencing, single cell sequencing, etc.

The tumor sample haplotypes node 712 (e.g., Hₕ) may be constrained by and connected by way of directed link to haplotype prevalence node 714

$$( e . g . , {Φ}_{h}^{d} )$$

of the DNA Dirichlet-Multinomial Model 704. In other words, Hₕ determines whether the prevalence node is non-zero.

$${Φ}_{h}^{d}$$

of the haplotype prevalence node 714 is the proportion of DNA molecules in the tumor sample which come from haplotype h. The system can estimate a set of haplotype prevalences by estimating

$${Φ}_{h}^{d} .$$

As discussed below, by fitting the model, the system can estimate a posterior distribution of

$${Φ}_{h}^{d} .$$

As discussed above and as depicted in the DNA Dirichlet-Multinomial Model 704,

$${Φ}_{h}^{d}$$

of the haplotype prevalence node 714 may be incorporated into the model and connected to the actual sequence reads that are observed via the following expression:

$$\begin{matrix}\begin{matrix}{p}_{m} = \frac{{\sum }_{hlja} I ( h , l , j , a ) \times {δ}_{a m}^{d} \times {ϕ}_{h}^{d} \times p ( {l}^{d} )}{{\sum }_{{hljam}^{′}} I ( h , l , j , a ) \times {δ}_{a {m}^{′}} \times {ϕ}_{h}^{d} \times p ( {l}^{d} )} \\ = \frac{{\sum }_{h} {W}_{m h} \times {ϕ}_{h}^{d}}{{\sum }_{{hm}^{′}} {W}_{{m}^{′} h} \times {ϕ}_{h}^{d}}\end{matrix} \\ \end{matrix}$$

where pₘ is the probability of observing a DNA sequence read that exhibits unique mutation pattern m.

$${W}_{m h}^{d}$$

(e.g., as represented by parameter 721) represents the unnormalized probability that, conditional on a DNA molecule being of haplotype h, a hypothetical DNA sequence read may exhibit a unique mutation pattern m and may be provided by the Enumeration and Pattern Probability Estimator 260.

pₘ (e.g., as represented by probability node parameter 718) represents the probability of observing a unique mutation pattern m. The probability node parameter 718 dictates, and thus is connected by way of directed link, to a DNA pattern counter node 720

$$( e . g . , {R}_{m}^{d} ) ,$$

in which the value

$${R}_{m}^{d}$$

may represent the observed values of DNA pattern counts present in the one or more samples, which can be provided by the read pattern counter 258.

RNA mutation patterns are modeled similarly to DNA patterns, but the system also accounts for different transcripts in the same gene. With reference to FIG. 7, the tumor sample haplotypes node 712 (e.g., Hₕ) may be further constrained by and connected by way of directed link to haplotype-transcript prevalence node 722

$$( e . g . , {Φ}_{ht}^{r} )$$

of the RNA Dirichlet Multinomial Model 706. In other words, Hₕ determines whether the prevalence node is non-zero.

$${Φ}_{ht}^{r}$$

in the haplotype-transcript prevalence node 722 is the proportion of RNA molecules in the tumor sample which come from haplotype h and transcript t. The system can estimate a set of haplotype-transcript prevalences by estimating

$${Φ}_{ht}^{r} .$$

As discussed below, by fitting the model, the system can estimate a posterior distribution of

$${Φ}_{ht}^{r} .$$

As discussed above and as depicted in the DNA Dirichlet-Multinomial Model 704, the estimated or approximated value

$${Φ}_{ht}^{r}$$

may be estimated or approximated based on the expression:

p(read has mutation pattern m, transcript group g)=

$$\begin{matrix}{p}_{gm} = \frac{{\sum }_{htlja} I ( h , t , l , j , g , a ) \times {δ}_{am}^{r} \times {ϕ}_{ht}^{r} \times p ( {l}^{r} )}{{\sum }_{{htljag}^{′} {m}^{′}} I ( h , t , l , j , {g}^{′} , a ) \times {δ}_{{am}^{′}}^{r} \times {ϕ}_{ht}^{r} \times p ( {l}^{r} )} \\ = \frac{{\sum }_{ht} {W}_{gmht} \times {ϕ}_{ht}^{r}}{{\sum }_{{htg}^{′} {m}^{′}} {W}_{{g}^{′} {m}^{′} ht} \times {ϕ}_{ht}^{r}}\end{matrix}$$

W^{r}_{gmht} (e.g., as represented by parameter 727) represents the unnormalized probability that, conditional on an RNA molecule being of haplotype h, and arising from transcript t, a hypothetical RNA sequence read from the molecule may exhibit a unique mutation pattern m and transcript group g—a quantity determined by the enumeration and pattern probability estimator 210.

p_{gm}_{^{r}} (e.g., as represented by parameter 724) represents the probability of observing a read exhibiting a combination of a unique mutation pattern m and a transcript group g. The parameter dictates, and thus is connected by way of directed link, to an RNA pattern counter node 726

$$( e . g . , {R}_{gm}^{r} ) ,$$

in which the value

$${R}_{gm}^{r}$$

may represent the observed values of mutation-pattern-transcript-group combination counts present in the one or more samples, which can be provided by the read pattern counter 258. The probability of observing a read pair with a given mutation and transcript group pattern can be dependent on the length of the nucleic acid fragment being sequenced (i.e. the “insert size”). Therefore, in order to determine the probability of observing a read pair with a given pattern (p_{gm} in the expression above), the system estimates the probability of a given fragment length (p(l) in the expression above). To accomplish this, the system extracts read pairs that align to long exons (e.g., exons that are longer than 200 bp, 300 bp, 400 bp, 500 bp, 600 bp, 700 bp, 800 bp, 900 bp, or 1000 bp) that do not overlap introns from other isoforms. In some embodiments, for example, the system can perform identification of long exons using the Python package pyranges, and can perform the identification of relevant read pairs using the Python package pysam. The absence of introns allows the read pair fragment lengths to be determined by inspection of their initial and final alignment coordinates (i.e., the start of the upstream read and the end of the downstream read). The system can model the resulting empirical distribution of fragment lengths using, e.g., a negative binomial distribution, which, in some embodiments, may be fit using the Python package statsmodels, the R package MASS, or similar tools that enable fitting of a negative binomial distribution to data. The final estimated distribution enables the determination of p(l) for any fragment length l.

The probability of observing a read pair with a given mutation pattern can be dependent on the probability with which nucleotide bases are misidentified in the sequencing data. For example, an erroneous base call could result in a read pair with mutation pattern “0, 1, 1” being observed in data from a sample containing only haplotypes “0, 0, 0” and “1, 1, 1”. The system therefore needs to estimate the probability, δₐₘ (see the expression above), that an observed pattern differs from the actual pattern. δₐₘ can be further decomposed into d, the number of incorrect base calls in an observed pattern, and ε, the probability of an individual incorrect base call, such that δₐₘ=ε^{d} (assuming that every type of miscall is equally likely). e can further be directly estimated from the data. In some embodiments, the system can perform this estimation by identifying all genomic positions across all sequence reads from a sample which lack a called mutation, and then looking for sequence reads that have a mutation that was not deemed a real mutation by the variant caller used. In some embodiments, the system can query alignments to these non-mutated positions using, e.g., the Python package pysam, the C utility samtools, or any other tool that facilities manipulation of aligned sequences, and can approximate the overall error rate F by the ratio of alignments that have the ‘incorrect’ (i.e. non-reference) base at those positions to the total alignments at those positions.

During use of the statistical model 262 in evaluating the sequence reads derived from a sample from a subject, the system can sample a set of possible haplotypes from a region of the genome of the subject. In some embodiments, the sampling can be done for the region from a healthy genome, for the region from a cancerous genome as provided by the tumor DNA sequences and the tumor RNA sequences, or a mixture of (i) the region from a healthy genome and (ii) the region from a cancerous genome as provided by the tumor DNA sequence reads and the tumor RNA sequence reads (e.g., sampling haplotypes present in either one of a sampling of the healthy genome or a sampling of the cancerous genome). In some embodiments, for each candidate haplotype in the set of all possible haplotypes, the value of an indicator variable denoting the haplotype's existence in the tumor tissue is sampled from a Bernoulli distribution having a beta prior.

With reference to FIG. 7, during the fitting of the statistical model (e.g., a probabilistic graphical model), each of parameters Xₕ (node 710), Hₕ (node 712),

$${Φ}_{h}^{d}$$

(node 714),

$${P}_{m}^{d}$$

(node 718),

$${Φ}_{ht}^{d}$$

(node 722), and

$${P}_{gm}^{d}$$

(node 724), can be sampled. Based on the sampling, the system estimate the posterior distributions for all six of these parameters. Accordingly, the system can estimate a posterior probability distribution of each haplotype prevalence, a posterior probability distribution of each haplotype-transcript prevalence, and a posterior probability distribution of the existence of each haplotype of the set of possible haplotypes. In some embodiments, the system uses Markov chain Monte Carlo (MCMC) sampling methods during the fitting process. In some embodiments, the estimated posterior probability distribution for a given parameter is derived from the sampled values for that parameter, across a plurality of samples (e.g., at least 500, 1000, 2000, 3000, 4000, 5000, 6000, 7000, 8000, 9000, or 10,000 samples). The sampling-based approach used in the disclosed methods (in contrast to other approaches that rely on determination of single point estimates of probability) readily provides a measure of the uncertainty in the estimates of

$${ϕ}_{h}^{d} and {ϕ}_{ht}^{r} .$$

With this uncertainty in hand, downstream neoantigen selection can penalize neoantigens when the uncertainty in estimated

$${ϕ}_{h}^{d} and {ϕ}_{ht}^{r}$$

is high.

In some embodiments, the statistical model may be a hierarchical Bayesian model. In some embodiments, the Bayesian analysis can be performed using algorithm(s) such as RJAGS or PyMC3. RJAGS provides an interface from R to the JAGS library for Bayesian data analysis.

### VI. Performance Evaluation

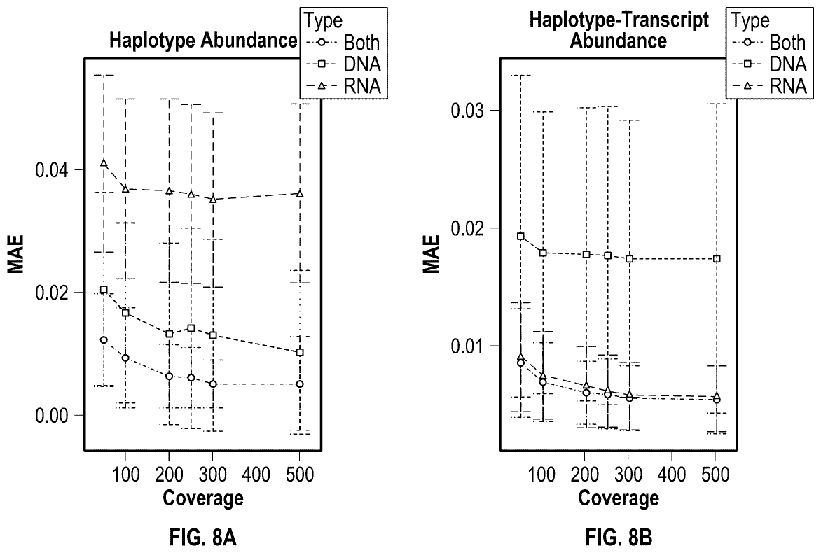

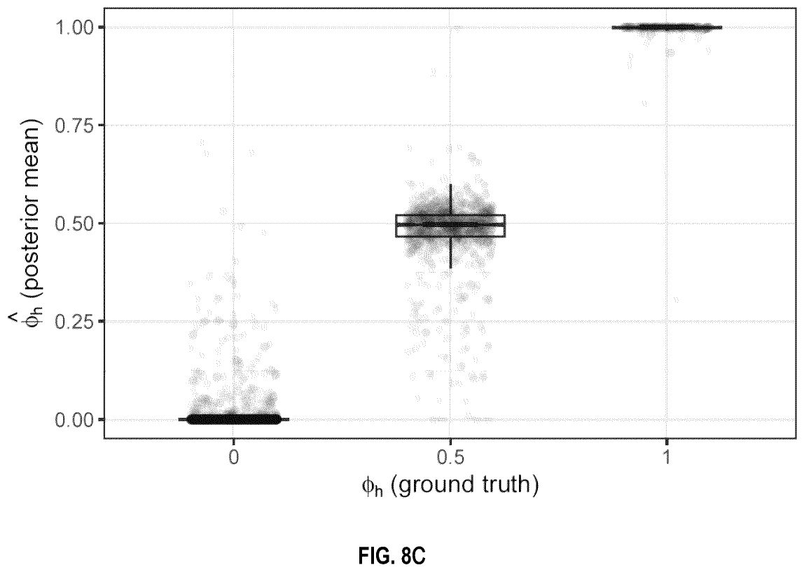

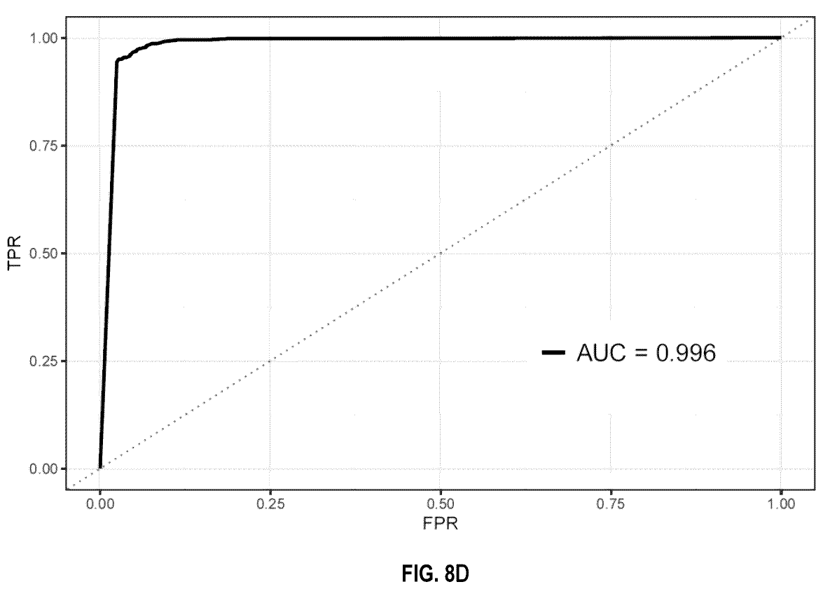

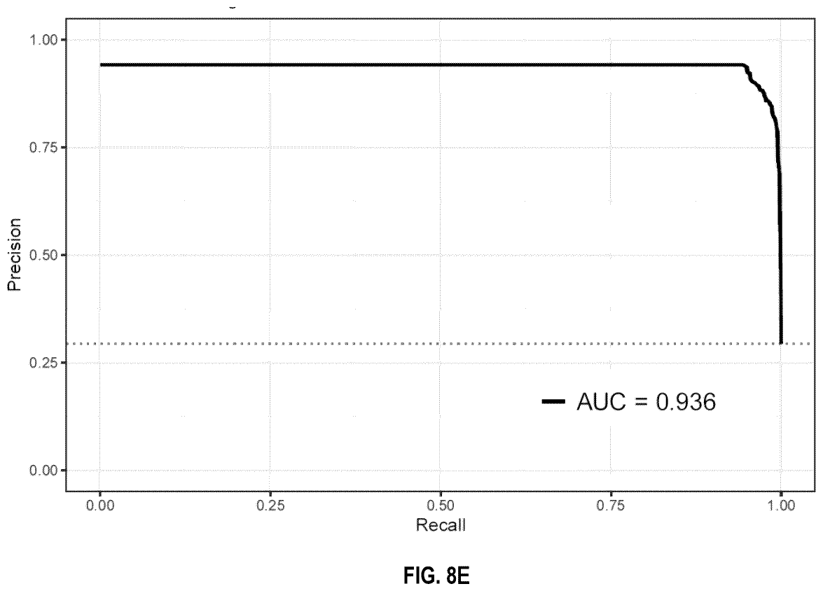

FIG. 8A illustrates a performance evaluation on simulated data of the techniques described herein. Specifically, performance solely of a probabilistic graphical model (PGM) (statistical model 262 in FIG. 2B and FIG. 7) portion of the system is evaluated here, by fitting the PGM to simulated read counts and simulated mutation positions, and then comparing the PGM outputs to simulated truth. This is in contrast to an end-to-end system evaluation on simulated read data—in which one would simulate phased mutations and then read data consistent with those mutations, align those simulated reads, and then apply the overarching system to the aligned reads, and compare the system's outputs to simulated truth.

The PGM evaluation process is described in more detail as follows:

Given a gene for which to evaluate PGM performance, an initial generative model randomly samples: (i) one of that gene's transcripts, and then (ii) variant positions landing in the exons of that transcript; the first variant position can be anywhere in any exon, and subsequent variant position are restricted to being within some distance of bases (in transcript space) of any already-sampled variant position. The variants themselves at these positions are specified to be biallelic, and are then provided as inputs to enumerate all possible haplotypes per gene.

With the set of mutations and all possible haplotypes in hand, along with a standard GTF specifying the possible transcripts for each gene, a second generative model—having almost identical structure to the PGM—was then employed to generate: (i) a simulated set of existing haplotypes—i.e., a subset of all possible haplotypes; (ii) the prevalence of each existing haplotype; (iii) each transcript-haplotype prevalence; (iv) the number of DNA reads for each mutation pattern; and (v) the number of RNA reads for each combination of mutation-pattern and transcript group.

There are two essential differences between this second generative model and the actual PGM 262. These are: (i) the process by which existing tumor haplotypes are sampled—rather than using a beta-binomial, we categorically sample P of the possible enumerated haplotypes to exist, where P is a user specified integer input, and (ii) the total number of reads is based on a user-input average read-coverage parameter, rather than coming from a bam file.

Once simulated truth data is generated, we apply the actual PGM (in RNA-only mode, DNA-only mode, or combined mode) to the simulated read counts, and estimate the haplotypes that exist, their prevalences and the haplotype-transcript prevalences. Point estimates specifically of these parameters (using a mean) are compared to the ground truth. The end-to-end process of simulated truth data and comparing it to estimates is repeated over many iterations of the simulation.

For FIG. 8A and FIG. 8B specifically, simulated data parameters were as follows: 4 mutations, read lengths of 100, maximum distance between mutations of 100, read coverage of 200×, P=4 haplotypes, and sequencing error probability of 0.01. 7 genes were evaluated: SNN, which has one transcript, DEFB125 which has two, MAP2K1 which has three, SMYD1 which has four, IAPP which has five, PPP4R4 which has six, and MCPH1 which has seven. The default number of iterations was 50.

In FIG. 8A, the mean absolute errors (MAE) for estimates of haplotype prevalence are plotted as a function of sequencing coverage for simulated sets of DNA sequence read data, RNA sequence read data, and a combination of DNA sequence read data and RNA sequence read data; we are comparing the performance of the model in its various modes. As noted elsewhere herein, the disclosed methods can be performed using DNA sequence read data (e.g., using models 702 and 704 in FIG. 7), with RNA sequence read data (e.g. using models 702 and 706 in FIG. 7) or with a combination of both (e.g., using models 702, 704, and 706 in FIG. 7). As shown, incorporating both RNA and DNA data reduces mean absolute error (MAE) in the estimation of haplotype abundance. Further, MAE is reduced as sequencing coverage (average coverage given the gene length) increases, leading to more accurate estimation of haplotype abundance. More broadly, this figure again shows that the model can in principle converge to a highly accurate solution for haplotype prevalences, and is not overparameterized given the amount of real data that we expect.

The data plotted in FIG. 8B is similar to that shown in FIG. 8A, with the difference being that it illustrates performance based on mean absolute error (MAE) for estimates of haplotype-transcript abundance. As shown in this figure, incorporating both RNA and DNA data reduces mean absolute error (MAE) of the estimation of haplotype-transcript abundance. Further, MAE is reduced as sequencing coverage (average coverage given the gene length) increases, leading to more accurate estimation of haplotype-transcript abundance. More broadly, this figure again shows that the model can in principle converge to a highly accurate solution for haplotype-transcript prevalence, and is not overparameterized given the amount of read data we expect.

FIG. 8C illustrates performance evaluation data for inferred values of φₕ that demonstrates close agreement with ground truth φₕ values in NA12878 genomic DNA, a diploid cell line having a previously haplotype-resolved genome. NA12878 sequencing data was downloaded from the NIH Sequence Read Archive (SRA): specifically, i) DNA normal tissue sequencing data, having ID SRR10134980, as generated by Illumina's NovaSeq 6000 with whole exome sequencing, comprising 242.3 million paired-end reads, with each end being 150 bp long; and ii) RNA normal tissue sequencing data, having ID SRR19762225, as generated by the Illumina NovaSeq 6000 with Poly-A mRNA sequencing, comprising 32 million paired-end reads, with each end being 150 bp long. Gold standard, haplotype resolved variant calls were download from the Illumina Platinum Genomes (with algorithms associated with these calls described in Eberle, M A et al. (2017), “A reference data set of 5.4 million phased human variants validated by genetic inheritance from sequencing a three-generation 17-member pedigree”, Genome Research 27: 157-164)

With normal (WES) DNA and normal RNA sequencing in hand, along with gold standard phased variant calls, we analyzed the data as follows: WES data was aligned to GRCh38 (Ensembl 100), using BWA-MEM2 to generate bams; RNA-seq data was also aligned to GRCh38, using STAR-2.7.9a; transcript expression was quantified using Salmon 1.3.0.

Next, treating normal DNA bams as if they came from tumor DNA, and treating the gold-standard germline variant calls as if they were somatic mutations, we applied the statistical model (as described in FIG. 7), specifically in its DNA-only mode, to predict Hₕ and φₕ^{d}. Phasing was restricted to those genes having at least 1 TPM as estimated by Salmon and having at least 10 RNA-seq reads; this filter was not strictly necessary, but, in the context of neoantigen characterization, lowly expressed genes are poor targets, and avoiding computation on these simply reduces runtime in a manner consistent with our expected use. We then compared the model-inferred haplotype prevalence (φₕ, determined as the mean of the posterior probability distribution) versus ground truth φₕ values for NA12878—this line being a diploid line, we take as a given that the ground truth prevalence for any haplotype that exists in the phased gold standard variant calls is either 50% or 100% prevalence, and for haplotypes missing from the gold standard data, the ground truth is 0% prevalence.

FIG. 8D illustrates performance evaluation data for using the disclosed methods to perform binary classification of haplotypes (i.e., a determination of whether or not a given haplotype is present, as estimated by mean Hₕ from the modelled samples) in NA12878 genomic DNA. NA12878 genomic DNA is derived from a diploid cell line having a known haplotype complement and thus known ground truth values for haplotype binary classification. The data for this non-limiting example includes all phasing windows comprising greater than 1 mutation and less than or equal to 6 mutations (i.e., phasing windows comprising 2, 3, 4, 5, or 6 mutations; trial runs also included phasing windows comprising 8, 9, or 10 mutations). True positive rate (TPR) was plotted against false positive rate (FPR) in this receiver operating characteristic (ROC) curve. True positive rate (TPR=TP/(TP+FN), where TP=# true positives and FN=# false negatives) and false positive rate (FPR=FP/(FP+TN), where FP=# false positives and TN=# true negatives) were computed at different H (posterior mean) thresholds for estimated haplotypes. The area under the curve was 0.996.

FIG. 8E illustrates further performance evaluation data for using the disclosed methods to perform binary classification of haplotypes in NA12878 genomic DNA, as described above for FIG. 8D. The data for this non-limiting example includes all phasing windows comprising greater than 1 mutation and less than or equal to 6 mutations (i.e., phasing windows comprising 2, 3, 4, 5, or 6 mutations; trial runs also included phasing windows comprising 8, 9, or 10 mutations). Precision was plotted against recall in this receiver operating characteristic (ROC) curve. Precision (=TP/(TP+FP)) and recall (=TPR=TP/(TP+FN)) were computed at different H (posterior mean) thresholds for estimated haplotypes. The area under the curve was 0.936.

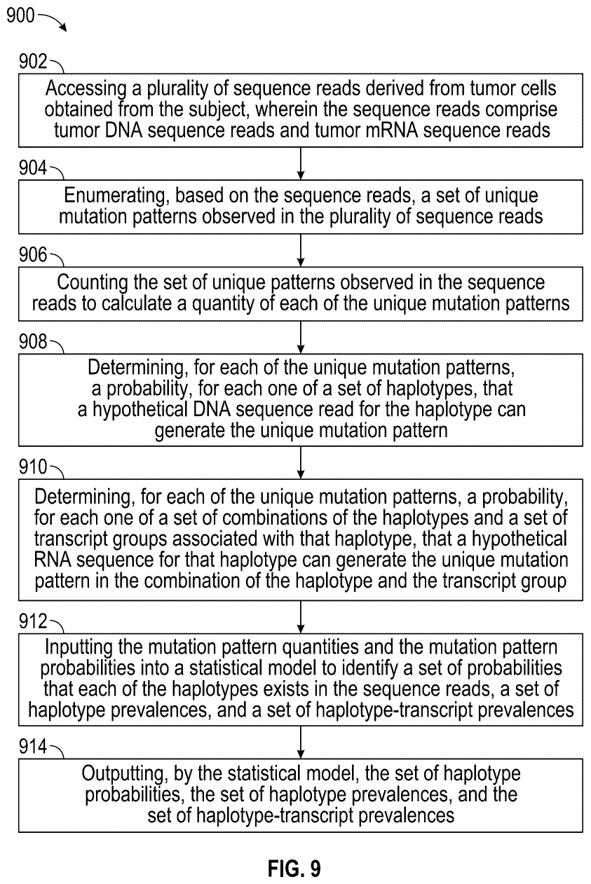

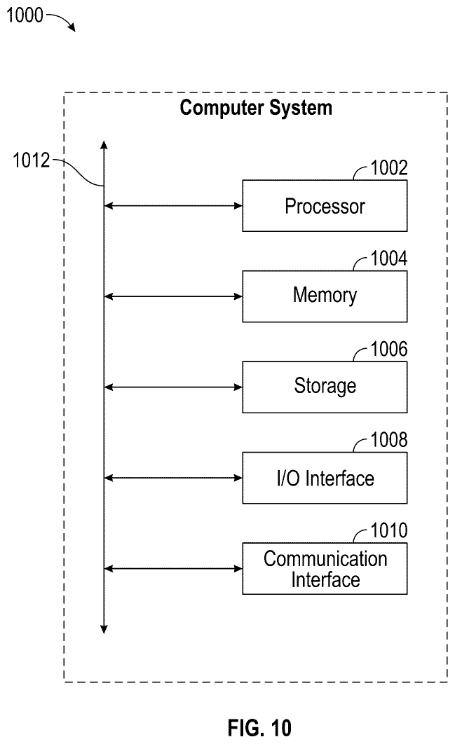

FIG. 9 illustrates a flow diagram of a method 900 for performing one or more computational-based methods for phasing mutations in a tumor of a subject, in accordance with the disclosed embodiments. The method 900 may be performed utilizing one or more processing devices (e.g., computing device(s) and artificial intelligence architecture to be discussed below with respect to FIG. 10 and FIG. 11) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other artificial intelligence (AI)/machine-learning (ML) accelerators device(s) that may be suitable for processing medical data and making one or more predictions or decisions based thereon), firmware (e.g., microcode), or some combination thereof.

In some embodiments, method 900 may begin at step 902. At step 902, a plurality of sequence reads derived from tumor cells obtained from the subject may be accessed, wherein the sequence reads comprise tumor DNA sequence reads and tumor RNA sequence reads. In one or more examples, accessing the sequence reads may further include accessing a plurality of normal DNA sequence reads derived from healthy cells obtained from the subject. In some embodiments, accessing the plurality of sequence reads further comprises accessing a set of germline and somatic variant calls derived from tumor cells obtained from the subject.

At step 904, a set of unique mutation patterns observed in the plurality of sequence reads may be enumerated based on the sequence read data.

At step 906, the set of unique patterns observed in the sequence reads may be counted to calculate a quantity of each of the unique mutation patterns. In one or more examples, counting the set of unique patterns may include calculating a quantity of each unique mutation pattern in the normal DNA sequence reads. In one or more examples, counting the set of unique patterns may include calculating a quantity of each unique mutation pattern in the tumor DNA sequence reads, and calculating a quantity of each unique mutation and transcript-group pattern in the tumor RNA sequence reads.

At step 908, for each of the unique mutation patterns, a probability, for each one of a set of haplotypes, that a hypothetical DNA sequence read from the haplotype can generate the unique mutation pattern may be determined.

At step 910, for each of the unique mutation patterns and transcript groups for each one of a set of combinations of the haplotypes and the transcripts overlapping the haplotypes, a probability that a hypothetical RNA sequence from that haplotype and that transcript will exhibit the unique mutation pattern in combination with the transcript group may be determined. In one or more examples, the probability that the hypothetical RNA sequence from that haplotype and transcript will exhibit the unique mutation pattern and transcript group in combination may further be based on a probability of miscalling tumor RNA sequence reads with the unique mutation pattern. In one or more examples, the probability that the hypothetical DNA sequence read from that haplotype will exhibit the unique mutation pattern may further be based on a probability of miscalling tumor DNA sequence reads with the unique mutation pattern. In one or more examples, the probability that the hypothetical RNA sequence from that haplotype and transcript will exhibit the unique mutation pattern and transcript group may further be based on a probability of an insert length for the RNA sequence read with the unique mutation pattern. In one or more examples, the probability that the hypothetical DNA sequence from that haplotype will exhibit the unique mutation pattern may further be based on a probability of an insert length for the DNA sequence read with the unique mutation pattern

At step 912, the mutation pattern quantities and the mutation pattern probabilities may be input into a statistical model to identify a set of probabilities that each of the haplotypes exists in the sequence reads, a set of haplotype prevalences, and a set of haplotype-transcript prevalences. In some embodiments, the estimation of haplotype-existence probabilities, haplotype prevalences, and haplotype-transcript prevalences comprises, for each haplotype of a set of candidate haplotypes, using the statistical model to: sample a posterior probability distribution for haplotype existence to determine a point estimate (e.g., mean probability) for haplotype existence, sample a posterior probability distribution for haplotype prevalence to determine a point estimate (e.g., mean probability) for haplotype prevalence, and sample a posterior probability distribution for haplotype-transcript prevalence to determine a point estimate (e.g., mean probability) for haplotype-transcript prevalence. In one or more examples, the set of possible haplotypes may comprise haplotypes that may exist in a healthy genome for the region. In one or more examples, the set of possible haplotypes may comprise haplotypes that may exist in a cancerous genome for the region. In one or more examples, the set of possible haplotypes may comprise haplotypes that may exist in a mixture of (i) a healthy genome for the region, as provided by normal DNA sequence reads and (ii) a cancerous genome for the region, as provided by the tumor DNA sequence reads and the tumor RNA sequence reads. In one or more examples, the set of possible haplotypes that may exist in the cancerous genome are sampled from a Bernoulli distribution using a Beta random variable. In one or more examples, the set of possible haplotypes that may exist in the mixture of (i) the healthy genome and (ii) the cancerous genome comprises haplotypes present in a healthy genome, or a cancerous genome, or in both.

At step 914, the set of haplotype probabilities, the set of haplotype prevalences, and the set of haplotype-transcript prevalences may be output by the statistical model.

In some embodiments, method 900 may include identifying a set of peptide sequences using translations of one or more haplotypes and/or haplotype transcripts based on the set of haplotype-existence probabilities. In one or more examples, the one or more haplotypes and/or haplotype transcripts are associated with non-zero probabilities.

In some embodiments, method 900 may further include a step of selecting one or more peptide sequences from the set of peptide sequences based on the set of haplotype prevalences and/or the set of haplotype-transcript prevalences. In one or more examples, the one or more peptide sequences may further be selected based on a predefined prevalence threshold. In one or more examples, the one or more peptide sequences may further be selected based on a ranking of the set of peptide sequences based on the set of haplotype prevalences and/or the set of haplotype-transcript prevalences. In one or more examples, method 900 may further include a step of identifying a set of mutant peptide sequences based on the one or more peptide sequences. In one or more examples, method 900 may further include a step of generating, by a machine-learning model, a prediction of a likelihood of presentation in a major histocompatibility complex (MHC) of one or more of the set of mutant peptide sequences or an immunogenicity of one or more of the set of mutant peptide sequences.

In some embodiments, method 900 may further comprise synthesizing one or more peptides (e.g., using one or more nucleic-acid sequences encoding the one or more peptides) or precursors to one or more peptides, where the one or more peptides comprise peptides (e.g., mutant peptides or neoantigens) selected on the basis of haplotype prevalences and/or haplotype-transcript prevalences determined using the methods described herein. The synthesized peptide(s) or precursor(s) may then be used in an experiment to identify corresponding presentation and/or binding data (e.g., to verify predicted presentation and/or binding). For example, an experiment may include assessing binding affinity of a selected peptide with a particular MHC molecule using an ELISA pull-down assay, gel-shift assays, or a biosensor-based methodology. As another example, an experiment may include collecting elution data indicative of whether a selected peptide was presented by an MHC molecule by using peptide-MHC immunoprecipitation, followed by elution and detection of presented MHC ligands by mass spectrometry.

In addition to or instead of verification data indicating whether individual peptides bound to and/or were presented by individual MHCs, verification data may indicate whether individual peptides triggered immunogenicity. Immunogenicity results may be determined using in vivo or in vitro testing. Testing the one or more selected peptides can be configured to investigate one or more immunogenicity factors (e.g., to determine whether and/or an extent to which a given event occurs) and/or immunogenicity (e.g., to determine whether and/or an extent to which the peptide triggers an immunological response). Testing can be configured to investigate whether administration of a composition (e.g., a vaccine) that includes one or more peptides to a given subject (e.g., for which an MHC sequence that was used during mutant-peptide selection has been identified) is effective in preventing or treating a medical condition (e.g., tumor) or disease (e.g., cancer). The subject may be a human subject.

In some embodiments, method 900 may further comprise designing and/or manufacturing a pharmaceutical composition (e.g., an individualized cancer immunotherapy and/or personalized anti-cancer vaccine) based on one or more selected mutant peptides corresponding to all or a portion of one or more neoantigens (or the design and/or manufacturing of a plurality of nucleic acids encoding the one or more selected mutant peptides). For example, each of the one or more selected mutant peptides may have been predicted to bind to and be presented by an MHC molecule of the subject (e.g., at least to a threshold degree). The pharmaceutical composition may include each of the one or more selected mutant peptides, one or more precursors to the one or more selected mutant peptides, one or more polypeptide sequences corresponding to the one or more selected mutant peptides, RNA (e.g., mRNA) corresponding to the one or more selected mutant peptides, DNA corresponding to the one or more selected mutant peptides, cells (e.g., antigen-presenting cells) including the one or more selected mutant peptides and/or nucleic acid(s) encoding such peptides, plasmids corresponding to the one or more selected mutant peptides and/or vectors corresponding to the one or more selected mutant peptides.

The pharmaceutical composition may further include an adjuvant, an excipient, an immunomodulator, a checkpoint protein, an antagonist of PD-1 (e.g., an anti-PD-1 antibody) and/or an antagonist of PD-L1 (e.g., an anti-PD-L1 antibody). The pharmaceutical composition may be a vaccine, such as a tumor vaccine. The composition may be an individualized vaccine manufactured or selected for a particular subject.

The pharmaceutical composition may include a polynucleotide construct (e.g., a DNA construct or an RNA construct). The polynucleotide construct is an artificially constructed segment of nucleic acid which may be ‘transplanted’ into a target tissue or cell. The polynucleotide construct comprises a DNA or RNA (e.g., mRNA) insert, which contains the nucleotide sequence encoding the one or more selected mutant peptides. In order to increase antigen presentation (e.g., presentation of the one or more selected mutant peptides by a MHC molecule), the polynucleotide construct may further comprise a modification developed for improved antigen presentation, and thus improved immunogenicity to the one or more selected mutant peptides. In some instances, the modification is incorporation of a transmembrane region and a cytoplasmic region of a chain of the MHC molecule into the polynucleotide construct as described in International Publication WO2005038030A1, which is incorporated herein by reference in its entirety for all purposes.

To provide an RNA insert with increased stability and translation efficiency, the polynucleotide construct may further comprise a modification developed for improved stability and translation, and thus improved immunogenicity to the one or more selected mutant peptides. In some instances, the modification is incorporation of a nucleic acid sequence with at least two copies of a 3′-untranslated region of a human beta-globin gene into the polynucleotide construct as described in International Publication WO2007036366A2, which is incorporated herein by reference in its entirety for all purposes. In other instances, the modification is incorporation of a nucleic acid sequence that codes for a 3′-untranslated region such as F1 3′ UTR described in International Patent Application Publication WO2017060314A3, which is incorporated herein by reference in its entirety for all purposes.

To provide an RNA insert with increased stability and expression, the polynucleotide construct may further comprise a modification developed for improved stability and expression, and thus improved immunogenicity to the one or more selected mutant peptides. In some instances, the modification is incorporation of a cap on an end of the RNA such as a 5′-cap structure. The cap structure may be the D1 diastereomer of beta-S-ARCA as described in International Patent Application Publication WO2011015347A1, which is incorporated herein by reference in its entirety for all purposes.

In order to deliver the polynucleotide construct with high selectivity to antigen presenting cells, the composition may further include cationic liposomes or a lipoplex for improved uptake of the polynucleotide construct, and thus improved immunogenicity to the one or more selected mutant peptides. In some instances, the composition includes nanoparticles comprising the polynucleotide construct. The nanoparticles may be lipoplexes comprising one or more lipids such as DOTMA and DOPE as described in International Patent Application Publication WO2013143683A1, which is incorporated herein by reference in its entirety for all purposes.

In some embodiments, as noted, method 900 may further comprise designing an immunotherapy and/or vaccine (e.g., an individualized cancer immunotherapy and/or personalized anti-cancer vaccine), where the immunotherapy and/or vaccine comprises one or more peptide sequences (e.g., neoantigen sequences) selected on the basis of haplotype prevalences and/or haplotype-transcript prevalences determined using the methods described herein.

In some embodiments, as noted, method 900 may further comprise manufacturing an immunotherapy and/or vaccine (e.g., an individualized cancer immunotherapy and/or personalized anti-cancer vaccine), where the immunotherapy and/or vaccine comprises one or more peptide sequences (e.g., neoantigen sequences) selected on the basis of haplotype prevalences and/or haplotype-transcript prevalences determined using the methods described herein.

Some embodiments of the disclosed methods further include treating a medical condition (e.g., tumor) or disease (e.g., cancer) in an individual by administering, to the individual, an effective amount of a pharmaceutical composition (e.g., an immunotherapy or vaccine) including one or more selected mutant peptides, where the one or more mutant peptides are selected using the methods disclosed herein. The individual may be the same individual from whom a disease sample was collected. In some instances, the vaccine is administered to a different individual as compared to the individual from whom the disease sample was collected. The different individual may, for example, be related to the individual from whom the disease sample was collected, have a genetic risk of developing a particular type of cancer, and/or have MHC molecules that have one, more or all alleles corresponding to a sequence that are the same (or similar) to one or more MHC alleles of the subject from who the disease sample was collected.

FIG. 10 illustrates an example of one or more computing device(s) 1000 that may be utilized to perform the techniques described herein, in accordance with the presently disclosed embodiments. In certain embodiments, the one or more computing device(s) 1000 may perform one or more steps of one or more methods described or illustrated herein. In certain embodiments, the one or more computing device(s) 1000 provide functionality described or illustrated herein. In certain embodiments, software running on the one or more computing device(s) 1000 performs one or more steps of one or more methods described or illustrated herein or provides functionality described or illustrated herein. Certain embodiments include one or more portions of the one or more computing device(s) 1000.

This disclosure contemplates any suitable number of computing systems 1000. This disclosure contemplates one or more computing device(s) 1000 taking any suitable physical form. As example and not by way of limitation, one or more computing device(s) 1000 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the one or more computing device(s) 1000 may be unitary or distributed; span multiple locations; span multiple machines; span multiple data centers; or reside in a cloud, which may include one or more cloud components in one or more networks.

Where appropriate, the one or more computing device(s) 1000 may perform without substantial spatial or temporal limitation one or more steps of one or more methods described or illustrated herein. As an example, and not by way of limitation, the one or more computing device(s) 1000 may perform in real time or in batch mode one or more steps of one or more methods described or illustrated herein. The one or more computing device(s) 1000 may perform at different times or at different locations one or more steps of one or more methods described or illustrated herein, where appropriate.

In certain embodiments, the one or more computing device(s) 1000 includes a processor 1002, memory 1004, database 1006, an input/output (I/O) interface 1008, a communication interface 1010, and a bus 1012. Although this disclosure describes and illustrates a particular computer system having a particular number of particular components in a particular arrangement, this disclosure contemplates any suitable computer system having any suitable number of any suitable components in any suitable arrangement. In certain embodiments, processor 1002 includes hardware for executing instructions, such as those making up a computer program. As an example, and not by way of limitation, to execute instructions, processor 1002 may retrieve (or fetch) the instructions from an internal register, an internal cache, memory 1004, or database 1006; decode and execute them; and then write one or more results to an internal register, an internal cache, memory 1004, or database 1006. In certain embodiments, processor 1002 may include one or more internal caches for data, instructions, or addresses. This disclosure contemplates processor 1002 including any suitable number of any suitable internal caches, where appropriate. As an example, and not by way of limitation, processor 1002 may include one or more instruction caches, one or more data caches, and one or more translation lookaside buffers (TLBs). Instructions in the instruction caches may be copies of instructions in memory 1004 or database 1006, and the instruction caches may speed up retrieval of those instructions by processor 1002.

Data in the data caches may be copies of data in memory 1004 or database 1006 for instructions executing at processor 1002 to operate on; the results of previous instructions executed at processor 1002 for access by subsequent instructions executing at processor 1002 or for writing to memory 1004 or database 1006; or other suitable data. The data caches may speed up read or write operations by processor 1002. The TLBs may speed up virtual-address translation for processor 1002. In certain embodiments, processor 1002 may include one or more internal registers for data, instructions, or addresses. This disclosure contemplates processor 1002 including any suitable number of any suitable internal registers, where appropriate. Where appropriate, processor 1002 may include one or more arithmetic logic units (ALUs); be a multi-core processor; or include one or more processors 1002. Although this disclosure describes and illustrates a particular processor, this disclosure contemplates any suitable processor.

In certain embodiments, memory 1004 includes main memory for storing instructions for processor 1002 to execute or data for processor 1002 to operate on. As an example, and not by way of limitation, the one or more computing device(s) 1000 may load instructions from database 1006 or another source (such as, for example, another one or more computing device(s) 1000) to memory 1004. Processor 1002 may then load the instructions from memory 1004 to an internal register or internal cache. To execute the instructions, processor 1002 may retrieve the instructions from the internal register or internal cache and decode them. During or after execution of the instructions, processor 1002 may write one or more results (which may be intermediate or final results) to the internal register or internal cache. Processor 1002 may then write one or more of those results to memory 1004.

In certain embodiments, processor 1002 executes only instructions in one or more internal registers or internal caches or in memory 1004 (as opposed to database 1006 or elsewhere) and operates only on data in one or more internal registers or internal caches or in memory 1004 (as opposed to database 1006 or elsewhere). One or more memory buses (which may each include an address bus and a data bus) may couple processor 1002 to memory 1004. Bus 1012 may include one or more memory buses, as described below. In certain embodiments, one or more memory management units (MMUs) reside between processor 1002 and memory 1004 and facilitate accesses to memory 1004 requested by processor 1002. In certain embodiments, memory 1004 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1004 may include one or more memory devices 1004, where appropriate. Although this disclosure describes and illustrates particular memory, this disclosure contemplates any suitable memory.

In certain embodiments, database 1006 includes mass storage for data or instructions. As an example, and not by way of limitation, database 1006 may include a hard disk drive (HDD), a floppy disk drive, flash memory, an optical disc, a magneto-optical disc, magnetic tape, or a Universal Serial Bus (USB) drive or a combination of two or more of these. Database 1006 may include removable or non-removable (or fixed) media, where appropriate. Database 1006 may be internal or external to the one or more computing device(s) 1000, where appropriate. In certain embodiments, database 1006 is non-volatile, solid-state memory. In certain embodiments, database 1006 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass database 1006 taking any suitable physical form. Database 1006 may include one or more storage control units facilitating communication between processor 1002 and database 1006, where appropriate. Where appropriate, database 1006 may include one or more databases 1006. Although this disclosure describes and illustrates particular storage, this disclosure contemplates any suitable storage.

In certain embodiments, I/O interface 1008 includes hardware, software, or both, providing one or more interfaces for communication between the one or more computing device(s) 1000 and one or more I/O devices. The one or more computing device(s) 1000 may include one or more of these I/O devices, where appropriate. One or more of these I/O devices may enable communication between a person and the one or more computing device(s) 1000. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1008 for them. Where appropriate, I/O interface 1008 may include one or more device or software drivers enabling processor 1002 to drive one or more of these I/O devices. I/O interface 1008 may include one or more I/O interfaces 1008, where appropriate. Although this disclosure describes and illustrates a particular I/O interface, this disclosure contemplates any suitable I/O interface.

In certain embodiments, communication interface 1010 includes hardware, software, or both providing one or more interfaces for communication (such as, for example, packet-based communication) between the one or more computing device(s) 1000 and one or more other computing device(s) 1000 or one or more networks. As an example, and not by way of limitation, communication interface 1010 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1010 for it.

As an example, and not by way of limitation, the one or more computing device(s) 1000 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the one or more computing device(s) 1000 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. The one or more computing device(s) 1000 may include any suitable communication interface 1010 for any of these networks, where appropriate. Communication interface 1010 may include one or more communication interfaces 1010, where appropriate. Although this disclosure describes and illustrates a particular communication interface, this disclosure contemplates any suitable communication interface.

In certain embodiments, bus 1012 includes hardware, software, or both coupling components of the one or more computing device(s) 1000 to each other. As an example, and not by way of limitation, bus 1012 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1012 may include one or more buses 1012, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, may be claimed in another claim category, e.g. system, as well. The dependencies or references back in the attached claims are chosen for formal reasons only. However, any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed comprises not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages

### EMBODIMENTS

Among the provided embodiments are:
1. A method for phasing somatic mutations and/or germline variants identified in a tumor of a subject, comprising, by one or more computing devices: accessing a plurality of sequence reads derived from tumor cells obtained from the subject, wherein the sequence reads comprise tumor DNA sequence reads and/or tumor RNA sequence reads; enumerating a set of unique mutation patterns observed in the plurality of sequence reads; counting a number of sequence reads that exhibit each unique mutation pattern of the set of unique mutation patterns observed in the sequence reads to calculate a quantity of each of the unique mutation patterns, and/or counting a number of sequence reads that exhibit each combination of a unique mutation pattern of the set of unique mutation patterns and a transcript group from one or more transcript groups associated with a gene to calculate a quantity of each combination of unique mutation pattern and transcript group; determining, for each of the unique mutation patterns, a probability, for each haplotype of a set of haplotypes, that a hypothetical DNA sequence read from the haplotype will exhibit the unique mutation pattern, and/or a probability, for each combination of a haplotype from the set of haplotypes, a transcript from a set of transcripts associated with the gene, and a transcript group from a set of transcript groups associated with the gene, that a hypothetical RNA sequence read from the haplotype and the transcript will exhibit the unique mutation pattern and the transcript group; inputting the unique mutation pattern quantities and/or unique mutation pattern-transcript group quantities, and the unique mutation pattern probabilities and/or unique mutation pattern-transcript group probabilities into a statistical model to estimate at least one of (i) a set of haplotype-existence probabilities that each of the haplotypes exists, (ii) a set of haplotype prevalences, and (iii) a set of haplotype-transcript prevalences; and outputting at least one of (i) the set of haplotype-existence probabilities, (ii) the set of haplotype prevalences, and (iii) the set of haplotype-transcript prevalences.
2. The method of embodiment 1, further comprising: identifying a set of peptide sequences using translations of one or more haplotypes and/or haplotype transcripts based on the set of haplotype-existence probabilities.
3. The method of embodiment 2, wherein the one or more haplotypes and/or haplotype transcripts are associated with non-zero probabilities.
4. The method of any one of embodiments 2 or 3, further comprising: selecting one or more peptide sequences from the set of peptide sequences based on the set of haplotype prevalences and/or the set of haplotype-transcript prevalences.
5. The method of embodiment 4, wherein selecting the one or more peptide sequences is further based on a predefined prevalence threshold.
6. The method of any one of embodiments 4 or 5, wherein selecting the one or more peptide sequences is further based on a ranking of the set of peptide sequences based on the set of haplotype prevalences and/or the set of haplotype-transcript prevalences.
7. The method of any one of embodiments 4-6, further comprising: identifying a set of mutant peptide sequences based on the one or more peptide sequences.
8. The method of embodiment 7, further comprising: generating, by a machine-learning model, a prediction of a likelihood of presentation in a major histocompatibility complex (MHC) of one or more of the set of mutant peptide sequences or an immunogenicity of one or more of the set of mutant peptide sequences.
9. The method of any one of embodiments 1-8, wherein accessing the sequence reads further comprises accessing a plurality of normal DNA sequence reads derived from healthy cells obtained from the subject.
10. The method of embodiment 9, wherein counting the set of unique patterns observed in the plurality of sequence reads further comprises: calculating a quantity of each unique mutation pattern in the normal DNA sequence reads.
11. The method of any one of embodiments 1-10, wherein counting the set of unique patterns observed in the plurality of sequence reads further comprises: calculating a quantity of each unique mutation pattern in the tumor DNA sequence reads; and calculating a quantity of each unique mutation and transcript-group pattern in the tumor RNA sequence reads.
12. The method of any one of embodiments 1-11, wherein the probability that the hypothetical RNA sequence from that haplotype will exhibit the unique mutation pattern in the combination of the haplotype and the transcript group is further based on a probability of miscalling tumor RNA sequence reads with the unique mutation pattern.
13. The method of any one of embodiments 1-12, wherein the probability that the hypothetical DNA sequence read from that haplotype will exhibit the unique mutation pattern is further based on a probability of miscalling tumor DNA sequence reads with the unique mutation pattern.
14. The method of any one of embodiments 1-13, wherein, the probability that the hypothetical RNA sequence from that haplotype will exhibit the unique mutation pattern in the combination of the haplotype and the transcript group is further based on a probability of an insert length for the RNA sequence read with the unique mutation pattern.
15. The method of any one of embodiments 1-14, wherein, the probability that the hypothetical DNA sequence from that haplotype will exhibit the unique mutation pattern is further based on a probability of an insert length for the DNA sequence read with the unique mutation pattern.
16. The method of any one of embodiments 1-15, wherein inputting the mutation pattern quantities and the mutation pattern probabilities into the statistical model to estimate the set of haplotype-existence probabilities, the set of haplotype prevalences, and the set of haplotype-transcript prevalences further comprises: sampling a set of possible haplotypes from a region of the genome of the subject; based on the sampling, estimating a posterior distribution, for the statistical model, of each haplotype prevalence; based on the sampling, estimating a posterior distribution, for the statistical model, of each haplotype-transcript prevalence; and based on the sampling, estimating a posterior distribution, for the statistical model, of the existence of each haplotype of the set of possible haplotypes.
17. The method of embodiment 16, wherein sampling the set of possible haplotypes comprises sampling haplotypes that may exist in a healthy genome for the region.
18. The method of any one of embodiments 16 or 17, wherein sampling the set of haplotypes comprises sampling haplotypes that may exist in a cancerous genome for the region.
19. The method of any one of embodiments 16-18, wherein sampling the set of haplotypes comprises sampling from haplotypes that may exist in a mixture of (i) a healthy genome for the region and (ii) a cancerous genome for the region as provided by the tumor DNA sequence reads and the tumor RNA sequence reads.
20. The method of any one of embodiments 16-19, wherein the set of haplotypes sampled from haplotypes that may exist in the cancerous genome are sampled from a Bernoulli distribution using a Beta random variable.
21. The method of any one of embodiments 16-20, wherein sampling from haplotypes that may exist in the mixture of the (i) the healthy genome and (ii) the cancerous genome comprises sampling haplotypes present in either one of a sampling of the healthy genome or a sampling of the cancerous genome.
22. The method of any one of embodiments 1-21, wherein accessing the plurality of sequence reads further comprises accessing a set of germline variant and somatic mutation calls derived from tumor and normal cells obtained from the subject.
23. A system including one or more computing devices, comprising: one or more non-transitory computer-readable storage media including instructions; and one or more processors coupled to the one or more storage media, the one or more processors configured to execute the instructions to perform any one of the methods of embodiments 1-22.
24. A non-transitory computer-readable medium comprising instructions that, when executed by one or more processors of one or more computing devices, cause the one or more processors to perform any one of the methods of embodiments 1-22.
25. A vaccine comprising: one or more peptides; a plurality of nucleic acids that encode the one or more peptides; or a plurality of cells expressing the one or more peptides, wherein the one or more peptides are selected from among a set of peptides by performing any one of the methods of embodiments 1-22.
26. A method of manufacturing a vaccine comprising: producing a vaccine comprising: one or more peptides; a plurality of nucleic acids that encode the one or more peptides; or a plurality of cells expressing the one or more peptides, wherein the one or more peptides are selected from among a set of peptides by performing any one of the methods of embodiments 1-22.
27. A pharmaceutical composition comprising one or more peptides selected from among a set of peptides by performing any one of the methods of embodiments 1-22.

What is claimed is:

1. A method for phasing mutations identified in a tumor of a subject, comprising, by one or more computing devices:
accessing a plurality of sequence reads derived from tumor cells obtained from the subject, wherein the sequence reads comprise tumor DNA sequence reads and/or tumor RNA sequence reads;
enumerating a set of unique mutation patterns observed in the plurality of sequence reads;
counting a number of sequence reads that exhibit each unique mutation pattern of the set of unique mutation patterns observed in the sequence reads to calculate a quantity of each of the unique mutation patterns, and/or
counting a number of sequence reads that exhibit each combination of a unique mutation pattern of the set of unique mutation patterns and a transcript group from one or more transcript groups associated with a gene to calculate a quantity of each combination of unique mutation pattern and transcript group;
determining, for each of the unique mutation patterns,
a probability, for each haplotype of a set of haplotypes, that a hypothetical DNA sequence read from the haplotype will exhibit the unique mutation pattern, and/or
a probability, for each combination of a haplotype from the set of haplotypes, a transcript from a set of transcripts associated with the gene, and a transcript group from a set of transcript groups associated with the gene, that a hypothetical RNA sequence read from the haplotype and the transcript will exhibit the unique mutation pattern and the transcript group;
inputting the unique mutation pattern quantities and/or unique mutation pattern-transcript group quantities, and the unique mutation pattern probabilities and/or unique mutation pattern-transcript group probabilities into a statistical model to estimate at least one of (i) a set of haplotype-existence probabilities that each of the haplotypes exists, (ii) a set of haplotype prevalences, and (iii) a set of haplotype-transcript prevalences; and
outputting at least one of (i) the set of haplotype-existence probabilities, (ii) the set of haplotype prevalences, and (iii) the set of haplotype-transcript prevalences.

2. The method of claim 1, wherein the estimation of at least one of the set of haplotype-existence probabilities, the set of haplotype prevalences, and the set of haplotype-transcript prevalences comprises:
(i) using the statistical model to sample from at least one of a haplotype-existence posterior probability distribution, a haplotype prevalence posterior probability distribution, and a haplotype-transcript prevalence posterior probability distribution for each haplotype of the set; and
(ii) calculating point estimates for haplotype-existence probability, haplotype prevalence, and haplotype-transcript prevalence for each haplotype of the set using the samples from the respective posterior probability distributions.

3. The method of claim 1, further comprising:
identifying a set of mutant peptide sequences using in silico translations of one or more haplotype sequences and/or haplotype transcripts based on the set of haplotype-existence probabilities, the set of haplotype prevalences, and/or the set of haplotype-transcript prevalences.

4. The method of claim 1, wherein the one or more haplotype sequences and/or haplotype transcripts are associated with non-zero haplotype-existence probabilities.

5. The method of claim 3, further comprising: selecting one or more mutant peptide sequences from the set of mutant peptide sequences using one or more predetermined criteria comprising a predetermined criterion that applies to the set of haplotype prevalences and/or the set of haplotype-transcript prevalences.

6. The method of claim 3, further comprising selecting one or more mutant peptide sequences from the set of mutant peptide sequences by ranking of the set of peptide sequences based on the set of haplotype prevalences and/or the set of haplotype-transcript prevalences.

7. The method of claim 3, further comprising:
generating, by a machine-learning model, a prediction of a likelihood of presentation in a major histocompatibility complex (MHC) for one or more of the set of mutant peptide sequences and/or a prediction of an immunogenicity for one or more of the set of mutant peptide sequences.

8. The method of claim 1, wherein accessing the sequence reads further comprises accessing a plurality of normal DNA sequence reads derived from healthy cells obtained from the subject.

9. The method of claim 1, wherein counting the set of unique mutation patterns observed in the plurality of sequence reads further comprises:
calculating a quantity of each unique mutation pattern in the tumor DNA sequence reads; and/or
calculating a quantity of each unique mutation pattern and transcript-group combination in the tumor RNA sequence reads.

10. The method of claim 1, wherein the probability that the hypothetical RNA sequence read from that haplotype and transcript will exhibit the unique mutation pattern and the transcript group is calculated from a haplotype-transcript prevalence, a conditional probability of observing the combination of unique mutation pattern and the transcript group in an RNA sequence read, and a transcript length.

11. The method of claim 1, wherein the probability that the hypothetical DNA sequence read from that haplotype will exhibit the unique mutation pattern is calculated from a haplotype prevalence and a conditional probability of observing the unique mutation pattern in a DNA sequence read.

12. The method of claim 1, wherein calculation of the probability that the hypothetical RNA sequence from that haplotype and transcript will exhibit the unique mutation pattern and the transcript group further comprises accounting for a probability of a given insert length for the RNA sequence read exhibiting the unique mutation pattern and transcript group.

13. The method of claim 1, wherein calculation of the probability that the hypothetical DNA sequence from that haplotype will exhibit the unique mutation pattern further comprises accounting for a probability of a given insert length for the DNA sequence read exhibiting the unique mutation pattern.

14. The method of claim 1, wherein calculation of the probability that the hypothetical RNA sequence from that haplotype and transcript will exhibit the unique mutation pattern and the transcript group further comprises accounting for an RNA sequencing error probability.

15. The method of claim 1, wherein calculation of the probability that the hypothetical DNA sequence from that haplotype and transcript will exhibit the unique mutation pattern and the transcript group further comprises accounting for a DNA sequencing error probability.

16. The method of claim 1, wherein estimating at least one of the set of haplotype-existence probabilities, the set of haplotype prevalences, and the set of haplotype-transcript prevalences comprises, for each haplotype of a set of possible haplotypes, using the statistical model to:
sample a posterior probability distribution for haplotype existence to determine a point estimate for haplotype existence;
sample a posterior probability distribution for haplotype prevalence to determine a point estimate for haplotype prevalence; and/or
sample a posterior probability distribution for haplotype-transcript prevalence to determine a point estimate for haplotype-transcript prevalence.

17. The method of claim 1, wherein accessing the plurality of sequence reads further comprises accessing a set of germline variant and somatic mutation calls derived from tumor cells obtained from the subject.

18. The method of claim 1, where the statistical model comprises a probabilistic graphical model.

19. A system including one or more computing devices, comprising:
one or more non-transitory computer-readable storage media including instructions; and
one or more processors coupled to the one or more storage media, the one or more processors configured to execute the instructions to:
access a plurality of sequence reads derived from tumor cells obtained from a subject, wherein the sequence reads comprise tumor DNA sequence reads and/or tumor RNA sequence reads;
enumerate a set of unique mutation patterns observed in the plurality of sequence reads;
count a number of sequence reads that exhibit each unique mutation pattern of the set of unique mutation patterns observed in the sequence reads to calculate a quantity of each of the unique mutation patterns, and/or
count a number of sequence reads that exhibit each combination of a unique mutation pattern of the set of unique mutation patterns and a transcript group from one or more transcript groups associated with a gene to calculate a quantity of each combination of unique mutation pattern and transcript group;
determine, for each of the unique mutation patterns,
a probability, for each haplotype of a set of haplotypes, that a hypothetical DNA sequence read from the haplotype will exhibit the unique mutation pattern, and/or
a probability, for each combination of a haplotype from the set of haplotypes, a transcript from a set of transcripts associated with the gene, and a transcript group from a set of transcript groups associated with the gene, that a hypothetical RNA sequence read from the haplotype and the transcript will exhibit the unique mutation pattern and the transcript group;
input the unique mutation pattern quantities and/or unique mutation pattern-transcript group quantities, and the unique mutation pattern probabilities and/or unique mutation pattern-transcript group probabilities into a statistical model to estimate at least one of (i) a set of haplotype-existence probabilities that each of the haplotypes exists, (ii) a set of haplotype prevalences, and (iii) a set of haplotype-transcript prevalences; and
output at least one of (i) the set of haplotype-existence probabilities, (ii) the set of haplotype prevalences, and (iii) the set of haplotype-transcript prevalences.

20. A non-transitory computer-readable medium comprising instructions that, when executed by one or more processors of one or more computing devices, cause the one or more processors to:
access a plurality of sequence reads derived from tumor cells obtained from a subject, wherein the sequence reads comprise tumor DNA sequence reads and/or tumor RNA sequence reads;
enumerate a set of unique mutation patterns observed in the plurality of sequence reads;
count a number of sequence reads that exhibit each unique mutation pattern of the set of unique mutation patterns observed in the sequence reads to calculate a quantity of each of the unique mutation patterns, and/or
count a number of sequence reads that exhibit each combination of a unique mutation pattern of the set of unique mutation patterns and a transcript group from one or more transcript groups associated with a gene to calculate a quantity of each combination of unique mutation pattern and transcript group;
determine, for each of the unique mutation patterns,
a probability, for each haplotype of a set of haplotypes, that a hypothetical DNA sequence read from the haplotype will exhibit the unique mutation pattern, and/or
a probability, for each combination of a haplotype from the set of haplotypes, a transcript from a set of transcripts associated with the gene, and a transcript group from a set of transcript groups associated with the gene, that a hypothetical RNA sequence read from the haplotype and the transcript will exhibit the unique mutation pattern and the transcript group;
input the unique mutation pattern quantities and/or unique mutation pattern-transcript group quantities, and the unique mutation pattern probabilities and/or unique mutation pattern-transcript group probabilities into a statistical model to estimate at least one of (i) a set of haplotype-existence probabilities that each of the haplotypes exists, (ii) a set of haplotype prevalences, and (iii) a set of haplotype-transcript prevalences; and
output at least one of (i) the set of haplotype-existence probabilities, (ii) the set of haplotype prevalences, and (iii) the set of haplotype-transcript prevalences.
